(19) United States
(12) Patent Application Publication
(10) Pub. No.: US 2006/0052341 A1
(43) Pub. Date: Mar. 9, 2006

(54) Control of a biological function

(76) Inventors: Brian Cornish, Hamilton (NZ); Andrew Philip Oakley, Hamilton (NZ)

Correspondence Address: Abelman Frayner Schwab, 10th Floor, 666 Third Avenue, New York, NY, 10017-5621, US

(21) Appl. No.: 10/504,236

(22) PCT Filed: Feb. 10, 2003
(86) PCT No.: PCT/NZ03/00018

§ 371 Date: Jul. 15, 2005

(30) Foreign Application Priority Data
Feb. 8, 2002 (NZ) 517093

(51) Int. Cl. A61K 31/724; A61K 31/56; A61K 31/557

(52) U.S. Cl. 514/58; 514/171; 514/573

(57) ABSTRACT

A biological function is controlled via the autonomous delivery of formulations administered at a single site, by determining each of the preferred formulations having efficacy in effecting control of at least one stage of a preferred biological function and includes features of improved permeation of formulations to effect desired bioavailability at a preferred level for a preferred period of time, one or more preferred formulations, in predetermined concentrations, in predetermined quantities, delivered at predetermined time intervals and over predetermined periods, and the delivery regimes for delivery of the formulations to achieve the outcome required. The formulations are delivered via a substance delivery device retained in location at a specific site for at least the delivery period. The device is adapted to house the formulations and the control and delivery apparatus required to effect controlled release of the formulations in accordance with the delivery regime.

### TECHNICAL FIELD

This invention relates to improvements in and relating to the control of a biological function.

In particular, the invention is directed to the autonomous delivery of formulations having efficacy in effecting a preferred biological function including aspects of improved permeation which may be required to effect desired bioavailability of at least active(s); to delivery regimes relating thereto; to apparatus for delivery thereof; to methods of manufacture and use associated therewith; and to a range of outcomes resulting therefrom.

For the purpose of describing the invention the discussion is directed to an example of controlling/synchronising/regulating oestrus in farmed or selectively bred animals, such as cows, sheep, pigs, deer, horses and so forth. The invention so exampled however, may have use in assisting breeding programmes for zoo animals or endangered species.

For the purpose of synchronising oestrus the formulations are administered at a single site, from a preferred delivery device located intravaginally, with controlled release of preferred quantities of the formulations (having preferred concentrations) into the animal occurring over a preferred period of time. The formulations for such a purpose are required to particularly directed to preferably having improved vaginal transmucosal permeation properties. Use of the formulations has the consequent advantage of convenience when artificial insemination and controlled breeding is practised. However, synchronising oestrus is also advantageous when managing stock populations generally.

Inherent in the use of formulations for synchronising oestrus is also therefore, the delivery regime for the introduction of the formulations in to the animal and the delivery apparatus/device used for this purpose.

It should be appreciated the present invention may also have applications outside this field. For example, aspects of the present invention may have application for the iin vivo release of other compounds, preparations and so forth, in a range of animals (including humans) to achieve a variety of outcomes. For example, nutritional, growth, drug delivery and anti-parasitic applications are but some of the alternatives. Whilst the formulations, delivery regimes and delivery device may be adapted for the particular application it is desirable that any formulations be directed to having improved absorption properties, enabling blood serum levels of the formulation compounds to be maintained at a preferred level for a preferred period of time to achieve the outcome required.

### BACKGROUND ART

The desirability to control a range of biological functions in animals (particularly mammals) has become an increasing feature of recent times. The requirement to do so has arisen from a need, actual or perceived, to regulate body processes to effect improved health, nutrition, reproductive capabilities and so forth. It is one object of the present invention therefore to provide autonomous delivery in situ of specific formulations via a specific delivery regime to effect control of a biological function.

The desirability to synchronise animal ovulation is but one example. It is increasingly required by farmers and reflects the farmers' management practices directed at maximising or optimising feeding objectives and resources, whilst at the same time enabling them to meet the ever changing demands of the market, both domestic and overseas. For optimum efficiency there is also a growing demand for single round synchrony programmes.

As knowledge of the oestrus cycle in domesticated animals, particularly herd animals such as cattle, has improved so has the ability to, manipulate the cycle for commercial reasons. For the purpose of this specification and to demonstrate the present invention, reference will be predominantly directed to synchrony programmes and formulations directed for manipulation of the oestrus cycle in cattle. However, this invention is also directed to address similar issues for a range of farmed animals, stud animals, or for use in breeding programmes for endangered animals. Again, as previously mentioned, the invention may also be adapted for a range of other applications.

Earlier synchrony programmes relied on the use/administration of one hormone to control a process that is naturally based on the in vivo interaction of many hormones. Early results therefore demonstrated either very effective synchrony of oestrus with low conception rates, or a spread in the onset of oestrus with normal conception rates.

Programmes using two hormones overcame these limitations to some extent but were still not sufficient to allow one fixed time insemination to a pre-planned time with normal fertility.

More recently, synchrony programmes using three hormones have demonstrated sufficient control over oestrus onset particularly in non-lactating cows or heifers to justify fixed time insemination. The hormones typically used include oestradiols, prostaglandins and progesterone. Increased knowledge has also enabled the encouragement of previously anoestrus lactating animals, such as beef or dairy cows, to come into oestrus with the ensuing oestrus being of higher fertility than that in the normal first oestrus after calving.

A number of formulations (including preferred hormones) and methods for introducing those formulations in to animals have subsequently been developed specifically directed to hormonal intervention in synchronising, controlling or preventing reproductive cycling, or even in hormone replacement therapies, with varying degrees of success.

For example, transdermal administration of progesterone, estradiaol esters and mixtures of these has been considered in EP 0279 977 A3. In this invention, a polymer matrix was used in which the drugs and a permeation enhancer were dispersed. However, this invention is directed at hormone replacement therapies rather than synchronising oestrus for managed breeding programmes and as such falls short of applicability in controlling a biological function, which is the focus of this application.

For programmes directed at synchronising oestrus in farmed and/or selectively bred animals, attempts to control the biological function highlighted problems of administering various actives/substances (hormones) to effect the desired outcome. For example, in some programmes some of the hormones (both oestradiols and prostaglandins) were and continue to be, injected intramuscularly for both ease of administration and efficacy of achieving desired blood serum levels of the active required. Whilst others (progesterone) tend to be administered through passive release systems via the vaginal route.

Internal administration of progesterone has been addressed in WO 89/02742 for example, where a tablet comprised of micronized progesterone was blended with carnuba wax and safflower oil. A sustained and substantially predictable increase in serum progesterone was achieved as a result of the in vivo rate of melting of the carnuba wax combined with the Imown rate of degradation of the progesterone by the liver.

In WO 97/37642 the administration of a medicament was disclosed. The medicament consisted of a biological medium-soluble capsule containing a micronised progesterone suspended in oil. As the capsule dissolved the progesterone was released. The capsule also contained estradiol enclosed in microspheres that consisted of polymer(s) that (although suspended in oil) did not dissolve in oil, but did dissolve in the biological medium.

Other licensed/existing products release hormone formulations on a passive basis that do not necessarily mimic the endogenous release of progesterone typically found in the fertility cycle. Improved bioavailability of the required hormones through the use of appropriate formulations and their release in situ at strategic times according to an optimal regime, will contribute to enhanced biological performance of such administered hormones. However, there is an ongoing need for the formulations released and/or methods used to be further developed to address problems of achieving good serum levels on a sustained and substantially predictable basis. It is a further object of this invention to make progress in this area.

It is also another object of this invention to provide an autonomous delivery system for formulations at one location in the body, rather than the previously used separate, often manual administration regimes delivering formulations at different sites. Single site release conversely provides the opportunity to effect autonomous delivery as a significant advantage over multi-site delivery options. Progress in the field has until now pot been directed to single site delivery of multiple actives using a specific delivery regime required to effect specific stages within the control of a biological function. As can be appreciated therefore, to effect the present invention raises a number of problems that separate site delivery does not.

Certainly, the intravaginal, transmucosal route for administration of hormones required to effect control of oestrus provides advantages over other administrative techniques in that it is a single site application, as opposed to having to administer one active intravaginally, another intramuscularly and so forth. If single site administration could be achieved so that it involved minimal re-handling of the animal, yet resulted in also obtaining required blood serum levels of the preferred hormones, then the actual formulations, the delivery regime employed and the delivery device used would contribute even more to the benefits that autonomous delivery offers. However, to effect such advances the need to further improve the permeation of the compounds through the vaginal mucosa becomes an issue. Controlled release of actives such as hormones and the permeation of them through the vaginal mucosal lining, to effect preferred blood serum levels, is extremely complex. To further manipulate a biological function which naturally requires the interaction of at least eight hormones over many days, also raises particular challenges.

In addition, the need also exists for a number of programmes suited to market demands for specific situations. Such programmes need to incorporate the most recent advances in animal breeding technology, be easy to administer and provide the assurance of consistent results. To effect the above applications it is therefore essential that, in any attempt to control a biological function, the formulations used need to be capable of inducing the required effect, they need to be delivered in a manner to ensure the required level in the blood is achieved as and when required and the process needs to be reliable.

In the area of controlling oestrus attempts have been directed to ensuring bioavailability of administered actives in situ. For example, where a medicament is administered in the form of a solution, a particular carrier solution may be used to assist the transfer of insoluble or partially soluble actives across membranes to effect the desired levels in the blood. For an active such as progesterone, benzyl alcohol has been a chosen carrier. This is because benzyl alcohol has the ability, to be saturated with high levels of progesterone (38-40% w/v) whilst remaining stable and without the progesterone precipitating out during storage or operation of delivery devices by which the solution is administered in to an animal. However, there are problems with the transportation of benzyl alcohol (particularly by air), and its usage may require approval of regulatory bodies in a number of countries.

Whilst there are a number of alternative carriers available some are more suited than others to effecting the transfer and bioavailability of a particular active, whether the active is a hormone, steroid, drug, nutritional supplement and so forth.

In describing the present invention and its application in controlling oestrus, the relevance of cyclodextrins as an alternative transmucosal carrier of hormone actives, requires discussion.

Cyclodextrins were first isolated by Villiers in 1891 as a digest from a bacteria culture on potato starch. The foundations for cyclodextrin chemistry were established during the early 1900s. However, up until 1970, only small quantities of cyclodextrins could be produced in the laboratory at extremely high costs. In more recent years, dramatic improvements in cyclodextrin production and purification have been achieved and a range of cyclodextrins (such as α-, β-, γ-cyclodextrins and their derivatives) have become much cheaper and more available. This has made industrial application of cyclodextrins possible.

The use of cyclodextrins as carriers to improve membrane transfer and bioavailability of partially soluble and/or insoluble actives is known. Cyclodextrins are capable of forming inclusion complexes with a wide variety of hydrophobic (whether totally insoluble or partially soluble) molecules by taking up a whole molecule, or some part of it, into the cyclodextrin ring. The stability of the complex formed depends on how well the guest molecule fits into the cyclodextrin.

Further, some cyclodextrins, such as β-cyclodextrin, are themselves only poorly water soluble. However, if its cyclodextrin derivative is used, such derivatives have demonstrated improved solubility and both the amount of active carried by it and by extension the solubility of the active itself, may be considerably increased. Common cyclodextrin derivatives are formed by alkylation or hydroxyalkylation of the hydroxyl groups or by substituting the primary hydroxyl groups with saccharides. These derivatives of cyclodextrin are used in a wide range of applications, primarily to allow solubilisation of sparingly soluble drugs and to improve the stabilisation of drug compositions.

A range of drug/cyclodextrin inclusion complexes have been manufactured for oral, parenteral and topical applications. Delivery of the inclusion complexes has been achieved by infusion, injection, drop, spray, aerosol, syrup, baths, tablets, suppositories, capsules, creams and ointments, for example. Intranasal and intraocular administration using nasal sprays and/or drops, enable the complex to be applied to and/or absorbed through the nasal and optical membranes. This, in turn, has successfully allowed transfer of the active ingredient via this membrane route. Nasal application of cyclodextrin complexes has also successfully demonstrated transfer of steroids such as oestradiol (used in hormone replacement therapy treatments).

The use of cyclodextrins as one of the range of suitable carriers of the actives of the present invention is an extension to both the earlier and more recent developments in this field.

Having regard to the above discussion therefore, the objects of the present invention are to provide a formulation(s) comprising, as required, particular substance(s)/active(s), with or without solvents, in a particular form, administered at a single site of release, via a preferred delivery regime involving single, multiple, or continuous doses, and where the doses are delivered in a preferred quantity, having a preferred concentration, and over a preferred time period. The formulation(s) preferably demonstrating where required, improved transmucosal permeation properties by use of carrier substances to achieve effective blood serum levels of the formulation(s) for the required time frames and the formulation(s) meeting the requirements of effectively controlling an aspect of the biological function—such as synchronising oestrus which is used as an example to describe the application of this invention in particular. Further that the delivery be effected using an appropriate delivery device capable of housing the necessary formulation(s) and being controlled to effect release of the formulation(s) as required via appropriate operation of componentry and that the device be comparatively easy to insert into the animal. Further, that the outcomes effected are preferably reliable and consistent.

Therefore, more directly, it would be advantageous to have formulation(s) and method(s) for delivering active compounds/components of the formulation(s) into an animal for the purpose of controlling/synchronising/regulating biological functions or stages thereof, such as ovulation and/or other biological cycles, that:
a) would make available preferred actives in required doses and/or at required times from a delivery device situated in the animal at a preferred site and delivered via a predetermined delivery regime to meet preferred requirements for synchronising/controlling a particular biological cycle, or a particular stage of a cycle, of a preferred animal; and
b) where the apparatus and method of use relating thereto provided one or more of the following benefits—was easy to use/introduce to the animal, effected reliable/consistent results, was tailored for use to the particular animal and/or biological function being controlled; and
c) where the formulation(s) comprised required active compounds in required doses that complemented the animal's normal biological functioning and/or effected required blood serum levels or other indicators associated with such biological functions; and
d) included the optional use of at least one alternative carrier directed at improving permeation and/or bioavailability of the active compounds/components of the formulation(s) and facilitating their introduction in required concentrations into the animal's system; and
e) did not need to include benzyl alcohol in the proprietary carrier solution for the active compounds/components; and
f) offered alternative carriers/solutions that the formulation's active compounds/components could be effectively carried by, and which could enable substantial increases in the quantity (on a weight:weight basis) of actives available in the body, over and above the levels often used in the prior art; and
g) offered a system whereby an alternative carrier/solution was able to deliver the actives in vivo via any one of being prior combined/dissolved with the active and then the combination being made available in powder, tablet, gaseous form or in solution and directly delivered in to the animal; and/or was able to be mixed with the active compounds/components (within a reservoir or simply at the delivery outlet at time of delivery) just prior to release into the animal and made available to the animal in liquid, powder, gaseous, or other form; and/or be released separately from, but in close proximity to, the active at the delivery site to effect combining after release in to the animal; and
h) offered a carrier/active combination that was one or more of stable, non-flammable, non-toxic; and
i) offered a delivery regime to deliver the formulations that was unique to the biological function being controlled; and
j) offered a delivery regime that was controllable; and
k) offered a delivery regime capable of effecting delivery of single unit doses, pulsatile doses and/or continuous dosing of the actives, singularly or in combination at a single in vivo site and/or delivering single or multiple actives, as required; and
l) offered a means via any one or more of the delivery regime and/or the carrier/solution and/or the ratio of active:carrier used, that was able to effect release of the active compounds/components (such as progesterone, to name but one) in vivo to meet the goal of elevating blood serum levels beyond a required threshold level to effect control and/or synchrony of the biological cycle or required outcome; and
m) offered a means to deliver an active, via either or both the delivery regime and/or the carrier/solution, that was capable of being delivered accurately from an in viva delivery device to meet the required delivery specifications; and
n) offered a means for controlling a biological function that was non-traumatic in all respects to the animal; and
o) offered an active/carrier formulation that was compatible with materials used in the delivery device used to deliver the formulations in vivo; and
p) offered a delivery device to effect the delivery, where the actives to be delivered could be retained in multiple reservoirs unique to the active and/or the specific dose of each active to be delivered; and
q) offered a delivery device to effect the delivery, where the delivery of each dose of any one active is effected by a delivery system specific to the form of the particular active and/or the dosing regime of the particular active; and
r) met requirements imposed by transportation of the formulation, and/or regulatory requirements set by authorities in a number of countries.

It is an object of the present invention to address at least some of the foregoing problems or at least to provide the public with a useful choice.

Further aspects and advantages of the present invention will become apparent from the ensuing description given by way of example only.

### DISCLOSURE OF INVENTION

According to one aspect of the present invention there is provided a method of controlling a preferred biological function of an animal, said method including the steps of:
a) determining the preferred formulations instrumental in effecting control of the biological function; and
b) determining the delivery regime required to effect release of one or more preferred formulation(s) of predetermined concentration(s), in predetermined quantity(s), at predetermined time(s) and over predetermined period(s); and
c) effecting delivery of the formulations in accordance with the preferred delivery regime from a substance delivery device, said delivery device being adapted to be retained in location for at least the delivery period, being adapted to house the formulations and including control and delivery apparatus to effect controlled release of the formulations in accordance with the delivery regime,
   the method characterised by effecting control of the biological function through the autonomous delivery of the formulations, from the delivery apparatus located in vivo, at a single site in the animal's body.

According to a further aspect of the present invention there is provided a series of formulations, each including at least one active component as herein defined, in combination with at least one facilitating transfer agent as herein defined, and optionally an excipient, the formulations compatible for delivery at substantially the same site during an administration regime, and which work in conjunction to achieve a particular physiological change associated with a biological function.

According to a further aspect of the present invention there is provided a series of formulations substantially as described above in which the biological function includes a reproductive process.

For the purpose of this specification the term facilitating transfer agent means any agent capable of facilitating transfer of an active or such like and shall include a penetration aid. Examples of a transfer agent include cyclodextrins or derivatives thereof, fatty acids, magnesioum, stearate, solvents, glycols and so forth. It should further be appreciated the term is not intended to be limited to only these said examples.

According to a further aspect of the present invention there is provided a series of formulations substantially as described above in which said formulations are suitable for delivery by a device of the type including a body, the body capable of housing delivery apparatus capable of actively being controlled to autonomously deliver at least one substance into a cavity, said delivery apparatus including dedicated pressure systems to deliver the formulations from independent reservoirs via associated outlet(s), said formulations ranging in form from substantially fluid to substantially solid, the device also including programmable control means capable of initiating and regulating delivery of the formulations in accordance with a preferred delivery regime, the body further including retention apparatus capable of effecting retention of the device within the cavity.

According to a further aspect of the present invention there is provided a formulation substantially as described above for in situ release in an animal including at least one active component as herein defined for affecting a biological function associated with reproductive processes, in combination with at least one facilitating transfer agent as herein defined, and optionally one or more excipients; said facilitating transfer agent including a cyclodextrin.

According to a further aspect of the present invention there is provided a formulation substantially as described above for in situ release in an animal including at least one active component as herein defined for affecting a biological function associated with reproductive processes, in combination with at least one facilitating transfer agent as herein defined, and optionally one or more excipients; said formulation compatible with at least a second formulation consisting of at least one active component as herein defined for affecting a biological function associated with reproductive processes, in combination with at least one facilitating transfer agent as herein defined, and optionally one or more excipients, for co-administration or co-delivery within the same administration regime duration at the same site; the two formlations being directed to achieve the desired outcome.

According to a further aspect of the present invention there is provided a formulation substantially as described above for in situ release in an animal in which the reproductive process is the synchronisation of oestrus.

According to a further aspect of the present invention there is provided a method for affecting a biological function using a formulation substantially as described above for in situ release in an animal consisting of automated release of active components, the method including an administration regime consisting of at least a first delivery phase for the release of at least a first active component, and a second delivery phase for release of at least a second active component, each phase consisting of parameters including one or more of release time, duration, magnitude; the release quantity versus time profiles on said two delivery phases differing, and wherein said first and second active components co-operate to achieved a desired outcome.

According to a further aspect of the present invention there is provided the control of a biological function by the concurrent operation of multiple delivery phases each directed to the release of a formulation including at least one active, the delivery being in situ and at substantially the same site, and delivered autonomously from a single arrangement in which one or more of the following parameters of release time, duration, magnitude is controlled in said regime.

According to a further aspect of the present invention there is provided reproductive processes controlled using a delivery device of the type including a body, the body capable of housing delivery apparatus capable of actively being controlled to autonomously deliver at least one substance into a cavity, said delivery apparatus including dedicated pressure systems to deliver the formulations from independent reservoirs via associated outlet(s), said formulations ranging in form from substantially fluid to substantially solid, the device also including programmable control means capable of initiating and regulating delivery of the formulations in accordance with a preferred delivery regime, the body further including retention apparatus capable of effecting retention of the device within the cavity.

According to a further aspect of the present invention there is provided delivery device of the type including a body, the body capable of housing delivery apparatus capable of actively being controlled to autonomously deliver at least one substance into a cavity, said delivery apparatus including dedicated pressure systems to deliver the formulations from independent reservoirs via associated outlet(s), said formulations ranging in form from substantially fluid to substantially solid, the device also including programmable control means capable of initiating and regulating delivery of the formulations in accordance with a preferred delivery regime, the body further including retention apparatus capable of effecting retention of the device within the cavity, programmed to implement a method of controlling a biological function.

According to a further aspect of the present invention there is provided an animal whose biological function is being controlled.

According to a further aspect of the present invention there is provided an animal whose biological function is being controlled through use of a device.

For the purposes of this specification reference to a substance delivery device used with this invention, particularly an intravaginal delivery device includes reference to the device as described in New Zealand Patent Application No. 517094 and PCT Application No. 96/00024.

It should also be understood that the term “comprise” where used herein is not to be considered to be used in a limiting sense. Accordingly, ‘comprise’ does not represent nor define an exclusive set of items, but includes the possibility of other components and items being added to the list.

This specification is also based on the understanding of the inventor regarding the prior art. The prior art description should not be regarded as being an authoritative disclosure of the true state of the prior art but rather as referring to considerations in and brought to the mind and attention of the inventor when developing this invention.

According to another aspect of the present invention there is provided at least one formulation for delivery into an animal for the purpose of controlling a preferred biological function, said formulation(s) capable of being delivered to the animal in vivo via a preferred delivery device, the formulation(s) including
a) at least one active compound in a dosage quantity capable of effecting the required change in at least one stage of the preferred biological function, and
b) a carrier capable of effecting absorption of the active(s) into the animal to achieve either or both a sustained and substantially predictable blood serum threshold level(s) required to effect the change needed to control the preferred biological function.

According to another aspect of the present invention there is provided a delivery regime for delivering preferred formulation(s) from a delivery device into an animal for the purpose of controlling a preferred biological function, said delivery regime including initiation and/or regulation of delivery in either or both in sequence and in unison, of one or more formulations from the delivery device to effect control of one or more stages of a known biological function, said delivery regime effecting release of the preferred formulation(s) of predetermined concentrations, in predetermined quantities, at predetermined times, for predetermined delivery durations and over an overall predetermined control period.

As can be appreciated, the present invention may be adapted to include a delivery regime whereby one formulation may be delivered from one or more outlets of the delivery device, for a varying lengths of time—from short duration to a prolonged period. Where there are multiple outlets delivering the same active, the concentration of the formulation may vary, additional compounds may be added to the separate formulations to effect delivery of these additives at precise times in combination with the active, or the duration of a particular delivery may be adapted to coincide with a particular stage of the biological function being controlled, and so forth.

Alternatively, a single formulation may be delivered from separate outlets at separate times, whilst at least one other formulation may be delivered from other outlets at the same time, or at timed intervals before, during or after delivery of the first or additional formulations. As can be appreciated the combinations are simply determined by the formulation(s) used and the biological function being controlled.

According to another aspect of the present invention there is provided a delivery regime for delivering preferred formulations into an animal for the purpose of controlling a preferred biological function substantially as described above wherein said delivery regime is effected via pre-programming and control via electronic control means included in the substance delivery device from which the formulations are released in vivo.

Use of programmable microchip(s), integrated circuits and so forth, typically associated with a preferred delivery device (that houses and is used to deliver the formulations in situ), enable the delivery regime to be targeted to deliver the preferred actives formulation(s), at preferred times, from specific reservoirs and outlets of the delivery device. The electronic componentry may be programmed to effectively turn on the overall delivery sequence, to regulate within the sequence individual aspects of the formulation(s) delivery such as the duration and/or outlet opening (and hence quantity of formulations delivered), to signal the endpoint of one delivery and the start of another, or to coincide delivery of one or more specific formulations as and when required, and so forth.

Accordingly, the formulations are able to be delivered according to a delivery regime required to best achieve the desired biological outcome. The regime will be predetermined and the delivery system pre-programmed and pre-calibrated to deliver the required formulations according to the required schedule. An example of a regime used to demonstrate this invention with reference to the management of oestrus in cattle is detailed later in this specification.

The regime and formulations to be delivered may vary from objective to objective. In one embodiment the regime may feature the active (as opposed to passive) initiation, active regulation, active control and active release of formulations required to control the preferred biological function on an autonomous basis. In another embodiment, the regime may feature the active initiation, active regulation, active control and active release of some formulations, with also the passive release of other formulations required to control the preferred biological function on an autonomous basis.

The regime may include any combination of one or more modes of delivery such as a single unit delivery of the formulations, as well as continuous, pulsatile or passive delivery modes.

The regime may also include delivery of the formulations in the form most suited to effecting transfer and/or bioavailability of the actives in the formulations as required. The same formulation may also be delivered in different forms at different times during the delivery sequence. Accordingly, the formulations may be delivered in substantially solid and/or substantially fluid form. As such the formulations may be delivered in gaseous form, as aerosol spray, liquid, solids, suspensions, pastes, micronised powders, solid capsules or tablets, and so forth, being the form most suited to the requirement of the delivery regime. For example, gaseous, liquid or more fluid forms may be suited to quicker and/or more thorough transfer, to effect rapid bioavailability. More solid forms or pastes and the like may be suited to slower transfer, reflected in a gradual increase in bioavailability over time (determined by the speed of transfer across membranes and so forth).

The form used is of course determined by the active and the biological function being controlled. At some stages, more immediate and/or high concentrations and/or ready availability of actives may be required. At other stages, slow build up of concentrations and/or delivery of levels over a prolonged period may be required. For example, the required delivery profile may mean that small amounts of actives are required at the beginning and end of the stage, with higher amounts in the middle of the stage. Slow release forms of the active may enable this process to be better controlled. Alternatively, rapidly available forms may be used, but delivery is controlled by the programmable regime to replicate the delivery otherwise available when slow release forms are used.

Accordingly, one distinct feature of this technology is, the ability to deliver single and/or multiple formulations to the target area from the one delivery system. A further distinct feature of this invention is the ability to initiate the delivery of particular formulations as and when required, via the same or different delivery modes to those employed for other formulations being delivered at the same or different times during the delivery regime. Yet further, the delivery regime and the delivery device used are both capable of accommodating the formulations in a variety of forms. For example, as mentioned previously, the formulations may be either in solid (tablet or capsule), liquid (including gels, solutions, sprays), suspension (pastes or forms having various viscosities) or gaseous form.

The formulations are capable of being stored in separate storage reservoirs within the delivery system and are then able to be delivered at the appropriate time according to the regime. Each formulation is capable of being delivered independently of another via its own unique separate delivery system contained within the device.

According to another aspect of the present invention there is provided at least one formulation for delivery into an animal substantially as described above wherein said formulation(s) is directed to improved vaginal membrane transfer of the active to effect preferred control of at least one stage of the biological function in the animal.

It should be appreciated however, that in attempting to control any biological function, there may be the need to deliver more than one formulation. For example, there may not be one formulation per se capable of improving vaginal transfer of a range of actives. Differing ratios of active:carrier or active:other formulation component(s) may be required depending on the actives (and/or carrier) used.

For ease of reference throughout this specification the preferred biological function used to demonstrate the invention, is the oestrus cycle. Specifically, the change in the oestrus cycle effected by the formulation(s) enables control of the cycle for the purpose of synchronising ovulation in animals for optimising farming practices and breeding programmes, particularly controlled breeding programmes. In this regard, the formulation(s) comprises hormones. However, as can be appreciated, the actives used to control another biological function may not necessarily include hormones.

It should also be appreciated that the term “control” used in this specification refers to the control, regulation, synchronisation, initiation and so forth of any biological function, stage thereof, or part of a stage thereof, by any means as described herein within the scope of this invention.

According to another aspect of the present invention there is provided a formulation(s) for delivery into an animal substantially as described above wherein said formulation(s) for effecting control of the biological function is actively introduced into the animal via use of a delivery device located at an appropriate site in or on the animal's body.

In the example directed to controlling oestrus, the formulations comprising hormones are preferably administered ini vivo via an intravaginal delivery device. The delivery device is adapted to include reservoirs for independently holding the formulation(s); means to effect activation, control, regulation and operation of the delivery device to enable release of the formulations to be undertaken actively and/or passively as required by the formulation delivery regime; means to effect retention of the device in/or on the animal for at least the period of delivery; and delivery means that when triggered effects release of the specific formulation(s) at the predetermined specific time and in specific quantity and/or to effect release of a preferred concentration, from a specific outlet, into a specific location in the animal.

The formulation(s) administered in vivo via insertion of an appropriately configured intravaginal device can be used in any lactating or non-lactating animals, although trials have predominantly focused on using dairy or beef cows and heifers. It should be appreciated that other preparations and/or hormone formulations may be administered to a range of animals for other purposes using a similar device, but where the device is adapted for use in a particular body cavity/release site specific to the purpose desired.

In the control of oestrus, a range of devices can be developed. Each device may be specifically tailored to one particular application of controlling oestrus in cattle. For example, devices should be able to provide the complex hormonal regime for one round of synchronised mating without a complicated implementation process characteristic of past controlled breeding programmes.

One product may be used to provide for single round synchrony suitable for use in either lactating or non-lactating, cycling or anoestrus, dairy or beef cows, whilst another may be suitable for use in providing single round synchrony in either dairy or beef heifers. Trials have demonstrated good results provided the planning, management and implementation of the programmes are carried out exactly to specification.

As can be appreciated there are advantages in having a delivery device that is a single administrative apparatus. These include the practical aspects of only having to introduce one apparatus into the animal, of being able to include all of the formulations required into one apparatus, the efficiency of having one device to control release from, and the lack of having to introduce formulations via other means such a suppositories, via injection, orally and so forth.

There are also advantages in the ability to release multiple actives at a single site. These include the ability to tailor the formulations to address practical physiological issues (such as transfer of the formulations across particular membranes because of solubility issues, and so forth) that may vary when different delivery sites are used—thereby requiring different carriers and/or different forms of actives, for example.

There are also advantages in the ability to control release of the actives to a predetermined regime. In this regard, the actives are automatically released relative to each other, rather than situations in the prior art where one active may be released passively from a device, whilst a second active is introduced in a different part of the body, such as via manual injection into different tissue. Where timing is critical to the efficacy of the control, independent delivery sites introduce the potential for errors that may detrimentally affect the outcome required.

According to another aspect of the present invention there is provided a formulation(s) substantially as described above wherein the active(s) are released in to the animal at a preferred site and according to a preferred and predetermined delivery regime, said formulation(s) being delivered from a delivery device including one or more independent reservoirs housing the formulation(s) and via one or more outlets.

The delivery regime is particular to at least the actives being used to control the particular biological function, the carrier to effect transfer and the biological function being controlled. Release of the formulation/actives at specific times and in specific amounts and/or concentrations and in specific form, is preferably predetermined and follows a particular pattern necessary to effect the desired outcome. For the purpose of controlling oestrus, one preferred delivery regime involves predetermined, controlled, active release of the formulation(s). The specific active/carrier formulations are released as either single unit doses or as pulsatile doses. In other embodiments, continuous flow or passive release may also be employed for some actives.

It must be appreciated that typically the delivery regime can include release of a combination of a variety of actives over time and that the delivery regime may include any combination of single unit doses, pulsatile doses, continuous flow or passive release that may be determined to be specific to the role of the active in effecting control of the overall, or a stage of, particular biological function.

According to another aspect of the present invention there is provided a formulation substantially as described above wherein the active(s) includes at least one of a drug, a hormone.

For ease of reference, the formulation(s), in the example for effecting control of oestrus, includes three reproductive hormones used as independent active compounds and at least one carrier enabling each of the hormones to be absorbed through the vaginal mucosa to maintain blood serum levels of the active required to effect the control of oestrus as required. However, it should be appreciated that this invention could be adapted to meet requirements for controlling other biological functions. For example, in other embodiments an active may be or include a nutritional supplement, a growth hormone, a parasitic treatment.

According to another aspect of the present invention directed to controlling oestrus, there is provided a formulation substantially as described above wherein the hormone active(s) includes at least one of progesterone, prostaglandin and oestradiol administered in preferred doses via the vaginal route to ensure effective transmucosal absorption of the hormone active required to obtain preferred levels of the hormones in blood serum for preferred periods.

According to another aspect of the present invention there is provided a formulation substantially as described above wherein the active(s) is available for use in a substantially solid and/or substantially fluid form. As such the active may be a powder; a gel, a liquid, a paste, a suspension of varying viscosities, a gas. The active(s) form may be determined by the requirements of dose required to be delivered into the animal over the preferred period of a preferred volume and/or a preferred concentration, the method of delivery appropriate to the quantity of active to be released, the physiology of the release site and as dictated by the constraints of the design of the delivery device. The active is any compound, chemical, hormone, mineral and so forth that is capable of effecting some physiological response in an animal, in any biological function or at any stage of a process thereof.

According to another aspect of the present invention there is provided a formulation substantially as described above wherein the active(s) are maintained in substantially dry form when mixed with a dry carrier, or dissolved/carried by a fluid carrier for, at or after, release of the formulation in vivo into the animal.

Whatever the carrier is, the active may be complexed with the carrier to form a specific premixed formulation; or the carrier may be present within the same (solid) formulation as the active but not complexed; or the carrier may be released as a separate substance at or about the same time as the active and is mixed during the release process; or the carrier may be released separately to the active but released in the same target location at or about the same time so that mixing of the carrier and active is enabled in the vicinity of the release zone.

According to another aspect of the present invention there is provided a formulation substantially as described above wherein the carrier includes any one of a fatty acid, a chemical compound.

According to another aspect of the present invention there is provided a formulation substantially as described above wherein the carrier is available for use in a substantially solid and/or substantially fluid form. As such the carrier may be in any of the following forms: a powder; a gel, a liquid, a paste, a suspension of varying viscosities, a gas.

In solid form the carrier may include a micronised powder. In liquid form the carrier may include a solution. Gaseous carriers may also be considered for use in this invention, but such use will be dependent on the active to be carried, the route of delivpry, the amount of active required, and so forth.

According to another aspect of the present invention there is provided a formulation substantially as described above wherein the carrier is a solution, said solution preferably being a solvent including at least one of water, an alcohol, other organic chemical solvents.

Water is a commonly used solvent for dissolving water soluble actives. However, many actives may be insoluble or only partially soluble in water. In such instances, other solvents (such as alcohols) may be used, or carriers that encapsulate/complex the active to improve its transferability across membranes.

According to another aspect of the present invention there is provided a formulation substantially as described above wherein the preferred alcohol carrier solution capable of carrying the active(s) in vivo includes at least one of benzyl alcohol, marlophen NP3, propylene glycol P1000, Ethanol and 2-phenylethanol.

Where the formulations are comprised of “dry” ingredients and little or no carrier solution may be present in the formulation, a preferred carrier for the formulations is cyclodextrin. Use of cyclodextrins enables the active material to be complexed for improved membrane transfer whilst cyclodextrins are useful for improving the transferability of substantially dry actives, it should be appreciated that cyclodextrins may also be added to liquid formulations in conjunction with the liquid solvent. For example, where water is a used as the solvent for the active, the formulation can also include cyclodextrin(s). The addition of cyclodextrin to benzyl alcohol used as a solvent (for progesterone for example) may also effect improved transfer of the hormone active as required for this invention.

A number of cyclodextrin derivatives have possible application to the current invention. There is significant prior art relating to the improvement of solubility and stabilisation of formulations containing the hormones that are discussed within the context of the current invention. On the basis of this prior art, various cyclodextrin derivatives (including gamma and beta cyclodextrins) have been considered in developing the formulations for effecting control of oestrus as described herein.

The preferred cyclodextrin for the formulations used for effecting control of oestrus in relation to the present invention is hydroxypropyl β cyclodextrin (HPβCD). However, other cyclodextrins and their derivatives may be used in formulations developed for controlling other specific biological functions. The choice of the specific cyclodextrin, its derivative and the final formulation is based not only on suitable efficacy but also on a range of practical considerations such as cost, availability of materials, stability of the formulation and ease of manufacturing the formulation.

The use of hydroxypropyl β cyclodextrin (HPβCD) is preferred for a number of reasons, not the least being the cost and availability of this derivative cyclodextrin, the efficacy of transfer and bioavailability of the actives when HPβCD is used—due to its solubility, the ease of handling, and so forth. Gamma cyclodextrin and/or a derivative thereof may also be used in other preferred embodiments. Gamma cyclodextrin is a more expensive option. Nevertheless, its efficacy may outweigh this factor.

The prior art has however indicated results from use of gamma cyclodextrin have not been as efficacious as those where HPβCD was used, making the cyclodextrin of choice in the prior art as HPβCD. However, results of tests conducted for the purpose of demonstrating the present invention have indicated inadequate weight:weight ratios of the active:cyclodextrin, such as oestradiol: HPβCD, or oestradiol:γ-cyclodextrin, appear to have been used in the prior art. As a consequence of the ratio values of the present invention, the efficacy of a range of cyclodextrins may be far more advantageous than previously considered.

The importance of selecting the most appropriate cyclodextrin and/or its derivative and ensuring the formulation contains an effective weight:weight ratio of active to cyclodextrin is well illustrated in existing prior art. In the prior art examples directed at the use of cyclodextrins with at least oestradiol, a low cyclodextrin-to-active ratio did not afford the optimal biological result and furthermore, there was inconsistency and variability in the rate of absorption and resulting metabolism of the active. Where control of a biological function is concerned, it is important for the rate and completeness of transfer, metabolism and bioavailability of the hormone actives (as in this case) to be effected reliably and consistently, through numerous/repeated/subsequent applications of the control process.

The example of the application of the present invention is directed to synchronising oestrus in herd animals in particular, as well as for stud/breeding animals. Such synchrony is used to facilitate among other things, controlled breeding programmes. It is again reiterated that whilst the ensuing description is directed at controlling oestrus in cattle, the invention can be adapted to species specific requirements of a number of mammals/animals, such as zoo animals and particularly “farmed” animals, including horses, sheep, goats, deer, llamas, pigs, ostriches and so forth, with possible application for breeding programmes for endangered species. Further, this invention is able to be adapted to release single or multiple actives in the form of drugs, food/nutritional supplements and so forth into an animal and may be adapted to control, regulate, synchronise and so forth other biological functions/cycles.

Reliability and consistency of results is therefore important for at least practical (resources of time, labour and money; business planning, breeding programmes, maintenance of livelihoods etc), ethical (animal health/husbandry, survival of species, etc) and marketing reasons (quality control, effectiveness of the product, etc), where a product to control a biological function is being used on herds of animals, endangered species, captive zoo animals and so forth.

Where the invention is particularly directed to controlling oestrus and the use of a strategic combination of the three hormonal actives discussed previously, it is important that the hormones are released at pre-determined times, to give sufficient control over oestrus onset in non-lactating cows or heifers to justify fixed time insemination.

Also, the invention has application with previously anoestrus lactating beef or dairy cows to control and/or synchronise the onset of oestrus with the ensuing oestrus being of higher fertility than that in the normal first oestrus after calving. Therefore, it is again important that the hormones delivered via this invention is able to give “non-cyclers” the opportunity to get in calf, as well as their cycling herd mates.

The following description is now directed to a discussion on the need for an understanding of the biological function being controlled and the problems required to be addressed. For example, in attempting to control oestrus, it is necessary to understand the pharmacology of the hormone actives, the form the active is used in, the carriers most suited to use with the particular hormone and the delivery regime to be implemented. There is a need to understand the physiology of the animal—for the design and function of any delivery device, for the implemented delivery regime of any actives and the type and form of carriers required to obviate problems due to the conditions in the anterior vagina (where the hormones are administered in the current example). It is also necessary to further consider regulatory requirements, animal health conditions, ease of use, ease of controlling delivery, ability to monitor results and reliability of results. As can be appreciated there are many further issues to consider. Additional ones will be known to those skilled in the art. Some of these issues have been discussed in part already. However, the ensuing description considers these issues more particularly from the perspective of actually controlling oestrus.

Pharmacologically, control of oestrus is complex, simply because the natural oestrous cycle itself is a complex series of hormonal interactions involving:
a) The hypothalamus which produces the peptide releasing hormone gonadotrophin releasing hormone (GnRH).
b) The anterior pituitary gland which produces the trophic protein hormones—lutenising hormone (LH) and follicle stimulating hormone (FSH).
c) The ovarian follicles which produce the ovarian steroid hormones androstenedione, oestradiol and progesterone.
d) The corpus luteum which produces high concentrations of progesterone as well as oxytocin.
e) The uterus which produces the prostaglandins.

In order to meet the goal of achieving substantially precise oestrus synchrony in cattle with good fertility through application of this invention it was determined necessary to use three hormones in combination (within the treatment period) and in specific concentrations. The approach chosen was found to more closely mimic the oestrous cycle, where in fact eight (8) hormones are interacting. Further, these three hormones are administered at strategic times for a specific end result.

The three hormones in the preferred embodiment discussed in relation to controlling oestrus in cattle are preferably an oestrogen, progesterone and a prostaglandin. In particular, in one embodiment described, the hormones are progesterone, oestradiol benzoate (oestra-1,3,5 (10)-triene-3,17β-diol 3-benzoate which, for ease of reference throughout the specification, will be discussed as oestradiol benzoate or OBD) and cloprostenol sodium. In yet another embodiment, the hormones used are progesterone, oestradiol hemihydrate (oestra-1,3,5 (10)-triene-3,17β-diol which, for ease of reference throughout the specification, will be discussed as oestradiol 17β) and cloprostenol sodium. Although, other actives may also be used instead of, or in combination with, the above.

When considering the several hormones produced during the naturally occurring oestrous cycle, each effects a particular activity in the animal. When the hormones are secreted in combination over time they produce the environment where oestrous activity is shown. With regard to the specific effects of each hormone available during theL ovarian cycle it is known that oestrogens induce:
a) proliferation of the vaginal epithelium,
b) increased secretion of mucous by the cervical glands,
c) endometrial proliferation; and
d) artesia of the non-dominant follicles.

The ovulatory follicle produces large amounts of oestrogen during the final maturation phase resulting in a positive feedback on the hypothalamus and pituitary at this stage of the cycle. This causes an elevation in gonadotrophin releasing hormone (GnRH) release that stimulates the secretion of lutenising hormone (LH). The lutenising hormone rise stimulates further oestrogen secretion from the follicular cells stimulating further lutenising hormone release and the resulting lutenising hormone surge, producing ovulation.

The higher levels of oestrogen cause behavioural oestrus that precedes ovulation. Oestrogen levels then decline rapidly as the period of “heat” progresses. At ovulation, which is 10-14 hours after the end of standing heat, oestrogen levels have returned to basal concentrations.

Three naturally occurring oestrogens have been found in the body. These are oestrone, oestriol and oestradiol. Oestradiol is the most important of the three naturally occurring oestrogens found in the body. Oestradiol and oestrone are freely inter-convertible. β-oestradiol (found in isolation studies from follicular fluid of sows' ovaries and from the urine of pregnant women) is the normally secreted ovarian hormone.

One of the presently disclosed programmes for synchronising oestrus in cattle relies on a twelve (12) day programme during which there is a preferred initial administration of oestradial benzoate, preferably as a spike at the start of administration of the progesterone active. Another preferred embodiment relies on a ten (10) day programme during which oestradiol 17β is administered, also as a spike. In both programmes the oestradiol is used to reset follicular waves by causing atresia of dominant ovarian follicles. This ensures the ovulatory follicle after ten, or eight days respectively (depending on the programme), of progesterone therapy is an actively growing healthy follicle producing an ovum consistently capable of being fertilised and initiating pregnancy.

Historically, oestradiol benzoate is often the most commonly used oestrogen. However, blood trials have supported the observation that there is poor absorption of oestradiol benzoate through the vaginal membrane and consequently poor metabolism of it by the liver before it becomes available in the preferred form for influencing oestrus. Consequently, systems relying on intra-muscular injections of oestradiol benzoate have in the past produced better results.

Further, oestradiol benzoate demonstrates hydrophobic properties. Accordingly, where it is delivered into the vagina, for example in tablet form, the oestradiol will be released in the region of the vaginal membrane, but rather than being promptly absorbed therethrough it leaches out of the tablet over an extended period of time. This delay is reflected in either or both the overall poor blood serum levels and the delay in reaching levels of oestradiol in the blood required to effect the desired outcome. Determination of an improved oestradiol formulation therefore led to the use of oestradiol 17β. However, similar vaginal transmucosal absorption problems can occur when oestradiol 17β is used. These observations are supported by blood trials.

Therefore, as the oestradiol must be processed by the liver before it is available for the purpose of controlled breeding programmes as addressed in the present invention, there is a requirement to facilitate transport of the oestradiol across the vaginal membrane as effectively as possible. Bearing in mind that oestrogens are not water soluble, there is a requirement for encasing the preferred oestrogen in a preferred water soluble carrier compound. One of the formulations described herein focuses on dissolving the oestradiol in alcohol or another solvent. In another preferred embodiment, the preferred carrier for the oestrogen component of the programme is at least one of a cyclodextrin, a suitable cyclodextrin derivative displaying the preferred properties, or a substitute compound displaying the preferred properties. It should be appreciated, that any other suitable carrier with the same or similar properties, that offers a cost-effective alternative to using cyclodextrin, may be used.

Using the formulations of the present invention, studies suggest that gamma cyclodextrin may be the most efficacious cyclodextrin form. However, as gamma cyclodextrin (and its derivatives) are very expensive, then the alternative, but also efficacious cyclodextrins include suitable derivatives from the beta family, such as hydroxypropyl-beta-cyclodextrin, dimethyl-beta-cyclodextrin and hydroxyethyl-beta-cyclodextrin, by way of example only. β-cyclodextrin on its own is poorly soluble in water and forms poorly soluble inclusion complexes. However, its derivatives, such as those mentioned above, are very water soluble and form very soluble inclusion complexes.

It should be appreciated that both the actives and the carrier compounds (in fact any compound) used with this invention may be derived from either or combination of natural or synthetic sources.

It has been recognised in the prior art that there has typically been poor vaginal administration of oestradiol benzoate. This poor performance has not been attributed to the dose, as typically doses are 10 times higher than those administered by intramuscular injection, but rather to the poor and variable absorption of the oestradiol benzoate following vaginal administration. Peak plasma oestradiol concentrations have been monitored at 2 to 5 pg/ml at between 2 to 48 hours after vaginal administration of 10 mg oestradiol benzoate. This is compared with peak plasma concentrations of 8 to 13 pg/ml obtained by approximately 2 hours following administration of 1 mg oestradiol benzoate administered by intramuscular injection.

Prior art attempts directed to effecting some control over oestrus have described the use of oestradiol 17β (although it is not clear whether this is the same form used as in the present invention) in conjunction with a carrier (cyclodextrin) as being more efficacious for intra-vaginal administration. However, the prior art focuses on the use of a ratio of the oestrogen active to cyclodextrin carrier administered intravaginally of less than 2:3 (active:carrier) by molecular amount and requires the serum levels of the active to be maintained for at least 24 hours in order to be efficacious. Accordingly, each dose has from 1.2-7.2 mg of oestradiol 17β (or 10-30 mg oestradiol benzoate) and 6-150 mg cyclodextrin(s). Using a dose of 7.2 mg of oestradiol 17β administered intravaginally prior art attempts have demonstrated peak plasma concentrations of between 10-20 pg/ml within four hours following administration, with the levels being elevated for at least 24 hours.

Further, it is reported in the prior art that vaginal administration of various amounts of oestradiol 17β (1.2, 2.5, 7.2 mg) with various molar ratios of γ-cyclodextrin (0.5:1, 1:1, and 3:2) to oestradiol 17β had no significant effect on the time to maximum plasma concentrations of the oestradiol.

However, trials conducted to determine the efficacy of the present invention with regard to the autonomous delivery of formulations, via a defined delivery regime, at a single site and delivered from a controlled delivery device, in, order to effect control of oestrus, identified values in relation to oestrogen delivery intra-vaginally (oestradiol 17β active: cyclodextrin carrier) needed to be substantially different from those of the prior art in order to be able to support the desired outcome.

While a range of doses of oestradiol (with a cyclodextrin carrier) has been identified for cattle breeding, the preferred dose of the oestradiol 17β active (as identified specifically in this specification) used for the current application is 2 mg. Nevertheless, as can be appreciated, different treatments may require different preferred doses of oestradiol. For example, the dose may fall within the range ≧0.5 mg to ≧7 mg of oestradiol encased in cyclodextrin carrier. The doses of oestradiol used may be influenced by improvements in the transferability of the oestradiol available from manufacturers and/or through developments in or efficiency of the carrier compound used and/or the form of the oestradiol and so forth.

Further, the half life of oestradiol benzoate via intravaginal delivery is generally longer than two to three minutes, as supported by the graphs relating to same herein. However, oestradiol benzoate is only one of the preferred oestrogen component of the present invention. In embodiments using oestradiol without the benzoate component, such as oestradiol-17β, the half-life can be significantly less. For example, the oestradiol 17-β form has a half life of only a few minutes. When oestradiol 17-β of the present invention is coupled with the preferred cyclodextrin carrier (which for the purpose of this discussion is hydroxpropyl 17β-cyclodextrin) a very rapid absorption is observed, with the corresponding peak in blood serum levels to achieve the desired results. Efficacious results have been obtained whether the oestradiol 17-β formulation is in solid (a tablet) or fluid form.

For the purpose of the present invention as exampled via the control of oestrus in cattle, release of oestrogen tablets into the vagina was carefully timed and recorded. Serum oestradiol rose rapidly from basal levels to reach peak values within 2 to 3 hours, with consistently well defined spikes. A 1 mg dose produced maximum mean values of 130-180 pg/ml at 100-130 minutes after treatment, while a 2 mg dose led to maxima of 180 to >250 pg at 120-150 minutes. The values attained exceed the peak plasma concentration maximums recorded in the prior art of 8 to 13 pg/ml obtained approximately 2 hours following administration of 1 mg oestradiol benzoate administered by intramuscular injection, or peak plasma concentrations of between 10-20 pg/ml within four hours following administration of 7.2 mg of oestradiol 17β administered intravaginally.

In determining the formulations for the present invention it was established that a primary objective was achieving a spike in plasma oestradiol to be an effective oestradiol surge for the purpose of stimulating (behavioural and/or functional) oestrus response. That the levels remain elevated for at least 24 hours, is not an important factor. Rather, a pronounced oestradiol spike (for short duration), total bioavailability, or period above a critical value, may be better correlated with clinical efficacy for either follicular atresia or stimulation of oestrus.

Concerns that excessive oestrogenic stimulation is likely is countered by the fact that no signs of excessive oestrous behaviour has occurred. It is likely that excessive stimulation is avoided because of the rapid clearance of oestradiol following treatment.

Accordingly, the formulation of the present invention is notably distinguished from prior art formulations in a number of features. Firstly, the formulation used to achieve the desired results relies on a ratio range of oestrogen active to cyclodextrin carrier administered intravaginally which may include 1:15 to 1:25 (active:carrier) weight for weight and is greater than previously used in the prior art. The range may even extend further from 1:8 to 1:35 (active:carrier) weight for weight, for example where different actives and different cyclodextrins or other carriers are used in the formulation. Secondly, the present invention requires the serum levels of the active to be maintained for only a relatively short period in order to be efficacious For example, a peak lasting approximately one hour appears to be more efficacious than elevated levels for at least 24 hours. Further, the present invention demonstrated peak plasma concentrations of between 130-180 pg/ml at 100-130 minutes after treatment with a 1 mg dose, while a 2 mg dose led to maxima of 180 to >250 pg at 120-150 minutes following administration. The duration of the peak and the peak plasma concentration may of course vary where the treatment is adapted for administration in different animals. Thirdly, the present invention administers two separate doses of the oestrogen (in one embodiment this is oestradiol 17-β) formulation, to effect the desired outcome. The first is preferably administered within approximately 2 hours following device activation, whilst the second is delivered on or about day nine (9) of a 10 day programme.

Importantly, it has been noted that increasing the amount of the oestradiol 17β, and/or increasing the amount of cyclodextrin within the preferred weight to weight ratio range of oestradiol:cyclodextrin does have a significant effect on the time to maximum plasma concentrations of the oestradiol. In addition there is a notable affect on the actual maximum plasma concentration of the oestradiol.

Progesterone is another hormone,relating to oestrus. Progesterone is a steroid hormone secreted by the luteal cells of the corpus luteum during dioestrus (day 5-18 of the cycle) and also by the placenta during pregnancy. The hormone is necessary for the preparation of the uterus for implementation of the fertilised oocyte and for the maintenance of pregnancy. The hormone acts upon the endometrium of the uterus, previously prepared by the oestradiol, inducing mucous secretion necessary for the implantation of the ovum. If pregnancy ensues, continued secretion of progesterone is essential for the development of the foetus until term. Progesterone secreted by the corpus luteum, rapidly builds up to a plateau by day 8 and is maintained until day 16. The high level of progesterone inhibits the final development and maturation of ovarian follicles via a negative feedback on the hypothalamus.

These high plasma concentrations of progesterone suppress the release of follicle stimulating hormone (FSH) and lutenising hormone (LH), the two gonadotrophines responsible for the final development and maturation of the dominant follicle, and prevent oestrous and ovulation. If the oocyte is not fertilised after ovulation, the uterus starts to release prostaglandin F2α from day 16. This has a luteolytic effect on the corpus luteum resulting in its rapid regression on days 17 or 18. Once the corpus luteum has regressed the plasma progesterone concentration drops to basal levels and a dominant follicle can mature and ovulate over the next few days.

Progesterone is rapidly metabolised in the body. For example, the half life of progesterone used in trials of the present invention has been approximately 20 minutes. In the urine, up to 30% of an administered dose appears as a conjugate of glucuronic acid and pregnanediol. Measurement of the urinary excretion of pregnanediol can be used to assess the rate at which progesterone is being secreted. Exogenous progesterone mimics that produced by the corpus luteum and creates the environment for continued hormonal interaction. The sudden fall in serum progesterone resulting from the removal of the exogenous supply is the switch leading to ovulation.

As a result of the relatively short half-life of the progesterone used with the present invention, the dosage amount and dosing intervals are accordingly determined to provide the most effective administration of the progesterone to the animal. For example, in embodiments where there is a 12 day delivery regime of the formulations overall, the progesterone is dosed for only 10 of those days. For a ten-day administration programme, the short half-life of the progesterone may mean that that progesterone is administered during 7-8 days rather than for the full 10-day programme (were the half-life of the progesterone greater). However, any dose regime is preferably established to ensure the dose frequency is not longer than the estimated half-life of the progesterone (of about 20 minutes).

Further, the intervals between the doses may also be reduced—for example, to approximately 30-35 minutes apart. The total volume of progesterone available for release into the animal, is of course, further limited by the dimensions of the reservoir within the substance delivery device. For example, in one embodiment as it relates to controlling oestrus, the progesterone reservoir may contain 10-12 mls of solution, whilst in another embodiment the reservoir may contain 40 mls of solution. The overall determination of dosage intervals and the total volume of progesterone available for release are of course ultimately controlled by the requirement to effect the preferred synchronisation and oestrus in the animal.

Progesterone is sparingly soluble. Some alcohols are effective at forming a stable solution. Some of the preferred solvents used in some embodiments of the present example of controlling oestrus, are discussed further in the specification. However, a large proportion of progesterone delivered using solvent carriers may not be readily absorbed by the vaginal mucosa. This in turn may lead to passive release following cessation of delivery. Variable effects identified in inconsistent blood serum results clearly identified that passive release of progesterone could not be included in an efficacious, planned dosing regime as required for the present example. Accordingly, in this regard the present example is distinguished from prior art treatments where passive release of progesterone is included in a delivery regime/treatment Nevertheless, in some embodiments for controlling a biological function, passive release of an active may effect a desired stage in the delivery regime.

To effect the desired control, it was identified as necessary to controllably elevate blood serum levels of progesterone by the introduction of an exogenous formulation including progesterone to effect a suitable elevation that was likely to prevent cows from “breaking through” into oestrus during treatment. Alternatives to alcohol were then identified with the specific objective of improving the bioavailability of the progesterone solution.

The objective for progesterone delivery was to achieve and maintain a preferred progesterone blood serum level for the duration of dosing which is 8 or 10 days in cattle (depending on the embodiment being referenced). It was a further objective that the level be targeted within 100 minutes of dosing commencing to effect coincidence in the delivery regime with the time of first oestradiol release. It was a further objective to also effect a rapid return to basal progesterone serum levels within 18-24 hours of cessation of dosing.

In preferred embodiments directed to controlling oestrus in cattle, the preferred range of progesterone in blood serum is 3-8 ng/ml within 100-min of dosing commencing. Whilst there is an acceptable minimum of 2 ng/ml, there is no maximum level, although beyond 8 ng/ml is deemed unnecessary. Nevertheless, in some larger animals, such as Buffalo, and so forth the upper range value may appropriately be increased.

Where there is no endogenous progesterone and dosing with exogenous progesterone is ceased, the preferred progesterone serum level should preferably be less than 1 ng/ml within 6 hours of progesterone dosing ceasing. The range would be from 0-2 ng/ml.

The dose range of progesterone with regards the present invention is preferably within the range between 0.5 gm to 2.2 gm. Further, the weight:weight ratio range of progesterone:carrier (cyclodextrin HPβCD) is preferably 1:8 to 1:17, with an optimum range of approximately 1:11 to 1:14. It should be appreciated however, that the dose and the ratios provided above are but one example. Accordingly, greater of lesser amounts may be dosed and the ratio of active:carrier may vary as required to effect the desired outcome.

Whilst the release of progesterone has been previously described with reference to the use of a liquid formulation, it should be appreciated that in yet further preferred embodiments the progesterone be in substantially solid form and may rely on body fluids along with the processes of dissolution and osmosis to effect substantially continuous delivery of the progesterone passively in to the environment around the substance delivery device. Given the challenge of delivering progesterone (as may apply to any other relevant substance) requires more frequent delivery, both the method of operation of the dose system and the formulation itself may be adapted as required. For example, the progesterone may be presented as a series of stackable tablets, or as a single block. The rate of dissolution may be controlled passively through the use of dissolution enhancing or dissolution limiting substances incorporated within the tablets or solid block at varying locations.

A plunger and/or spring system may be used to urge the solid form of progesterone to a location at the surface of the device such that the solid form is presented in a position to effect optimum delivery of the substance where it may be eroded away.

This passive delivery using a solid form of the substance may be used alone or in conjunction with a simultaneous, continuous and/or periodic release of a substantially fluid form of the same substance, as may be required to effect the desired outcome.

With all of the formulations discussed herein, improved bioavailability of the active through use of improved carrier formulations may enable the quantity of the active used to be decreased, yet still effect the desired outcome. Alternatively, where the affect is required to be significant and that affect may be effected by increased quantity of active delivered, then improved carrier formulations may assist this desired outcome also.

The third preferred active used in the exampled embodiment is a prostaglandin. Prostaglandins are metabolites of the arachidonic acid cascade. The principal, biological active, naturally occurring prostaglandins are prostaglandin E₂ (PGE₂), prostaglandin F_{2α} (PGF_{2α}), prostacyclin (PGI₂) and thromboxane (TXA₂).

They are very potent agents and have various local biological actions. Their half-life is short due to their rapid breakdown—a few minutes for prostaglandin PGF_{2α}.

Regression of the corpus luteum (CL) in cycling cows is caused by pulses of prostaglandin (PG) being secreted by the endometrium of the uterus. These pulses are facilitated by oestradiol from the ovarian follicles in mid to late di-oestrous. Oestradiol acts by stimulating the formation of oxytocin receptors in the endometrium. These receptors are activated by oxytocin secreted by the corpus luteum resulting in protaglandin release and luteolysis.

In a pregnant cow prostaglandin is released at the end of gestation signalling the beginning of contractions of the uterus and cervix to allow for the expulsion of the calf.

In the present application using the example of synchronising/controlling oestrus, the prostaglandin is required to be administered at a point in advance of the cessation of exogenous progesterone administration to, ensure endogenous progesterone will not mask the precipitous fall in serum progesterone resulting from this cessation. Prostaglandin is required as a luteolytic to remove any endogenous progesterone and ensure control of the progesterone drop is only the result of cessation of the exogenous supply. As this is administered in the present delivery regime on approximately day 7.5 when there has been sensitisation of prostaglandin receptors, only half the recommended therapeutic dose of prostaglandin is required to be luteolytic. In other situations/regimes the day of administration of the prostaglandin may differ to that described above.

Within the prior art, discussion has focused on the lack of efficacy in delivering sodium cloprostenol intravaginally when compared with intramuscular applications. Whilst various rates were assessed no carrier was used to effect improved transfer/absorption through the vaginal mucosa. Sodium cloprostenol is similar to progesterone in that it is sparingly soluble in water.

In effecting the desired delivery regime for the present invention as directed to use in controlling bestrus in cattle, a number of studies were conducted. It was identified that use of sodium cloprostenol without a suitable carrier (preferably a cyclodextrin) was not having a complete luteolic effect at the level of sodium cloprostenol dosed. However, incomplete luteolysis was also noted where the same dose was administered with HPβCD. Increasing the dose of sodium cloprostenol demonstrated a dramatic improvement in the speed of metabolism and clearance of exogenous progesterone and this resulted in corresponding improvements in expression of overt oestrus and ovulation.

Further, in the present invention, the benefits of using cyclodextrins such as HPβCD, as a carrier for the prostaglandin sodium cloprostenol in conjunction with the preferred increased dose rate, has demonstrated an equivalent response to treatment in animals as found in prior art research results using intramuscular injection of prostaglandin alone.

The affect of increased solubility and the higher progesterone level (which contributes to the prostaglandin receptors being more receptive) may also contribute to the improved efficacy.

There is no reliable blood test for sodium cloprostenol. The best test is to assess the success of luteolysis following application of sodium cloprostenol. If luteolysis is successful then there will be little or no endogenous production of progesterone immediately following application of a suitable dose of sodium cloprostenol (for example, within 6 hrs). Progesterone blood serum levels should preferably be below 1 ng/ml (providing there is no exogenous supply of progesterone still remaining) thus providing a useful indicator.

The recognised therapeutic dose of sodium cloprostenol is 500 μg. However, in preferred embodiments of the present invention as directed to the example of controlling oestrus in cattle, the preferred dose range for sodium cloprostenol is between 500 μg and 1.5 mg per unit dose when combined with a preferred carrier (such as cyclodextrin HPβCD). Nevertheless, in heifers it is possible that as little as 250 μg sodium cloprostenol plus HPβCD may be sufficient, or similarly for larger cattle species such as buffalo a rate of ≧2.5 mg may be required for optimal results.

The weight:weight ratio range of prostaglandin:carrier (cyclodextrin HPDCD) is preferably 1:7 to 1:18, with an optimum range of approximately 1:11 to 1:14. It should again be appreciated however, that the dose and the ratios provided above are but one example. Accordingly, greater of lesser amounts may be dosed and the ratio of active:carrier may vary as required to effect the desired outcome in different animals, using different carrier derivatives and different forms of the active hormones.

Whilst there has been reference above to improved transfer of the actives for bioavailability in blood serum, the following discussion reiterates and/or adds to the information previously provided with respect to various carriers in various forms.

For example, in one preferred embodiment of the present invention, some, if not all the hormone actives may be dissolved in solution prior to release into the animal, to ensure complete and rapid uptake through the vaginal mucosa. This is preferably the case for progesterone. In one preferred embodiment, the solvent in which the hormones are dissolved is preferably alcohol based, although water may be used in other embodiments, alone or in conjunction with any other preferred solvent.

Early developmental trials used benzyl alcohol as a preferred carrier. For example, as a carrier of progesterone, it has the ability to be saturated with high levels of progesterone (38-40% w/v) and remain stable, without the progesterone precipitating out during storage or during operation of delivery devices (that administer the progesterone solution) in an animal.

However, problems with the transportation of benzyl alcohol (particularly potential hazards when transporting benzyl alcohol by air), and the probable requirement for regulatory approval in a number of countries, prompted assessment of alternative carrier solutions. Whilst benzyl alcohol can be used with the formulations, preferred carriers now also include marlophenol, propylene glycol and phenylethanol, ethanol (typically 70%-99.8%) and water.

Further, the applicant has also developed a method for effecting improved transfer of the progesterone through the vaginal mucosa, by the use of a fatty acid as a penetration aid. Replacement of a percentage volume of the solvent (approximately 33%) with a preferred fatty acid, not only assists in the transfer of the progesterone across the vaginal mucosa, but is also a cost-effective option. Trials conducted by the applicant have demonstrated favourable results.

Similarly, incorporation of magnesium stearate as a penetration aid/carrier in a tabletised form for the prostaglandin active has been found to assist penetration of the unit doses of the prostaglandin through the vaginal mucosa.

Whilst the hormones can be dissolved in the preferred alcohol solutions mentioned previously to ensure improved, maximum and rapid uptake through the animal's vaginal mucosa, other options are available. For example, alternatively, or in combination with the solvents, other carriers as penetration aids may also be used. These include cyclodextrins as previously discussed.

For the purpose of demonstrating this invention using control of the oestrus cycle as an example, it was necessary to determine the most appropriate carrier, the most appropriate ratio of active to carrier, the most appropriate form of the carrier:active delivered, and the most appropriate delivery regime for each formulation. This necessitated an understanding of prior art delivery regimes, formulations, sites of delivery and delivery vehicles.

Preferred programme strategies for the present invention rely on the combination of hormones including progesterone, a preferred form of the oestradiol and cloprostenol sodium in conjunction with their preferred carrier, in solution or in tabletised form, delivered intravaginally. The results are consistent with prior art synchrony work on 50,000 cows and heifers using 1 ml estrumate (240 mcg cloprostenol—Mallinckrodt) that also demonstrated consistent results in both cows and heifers.

Previously however, prior art treatment of the non-cycling dairy cow used a progesterone releasing silastic device followed by intramuscular administration of 400 IU equine chorionic gonadotrophin at time of device removal 5 to 8 days later.

This prior art combination treatment in lactating cows that had calved more than thirty days previously resulted in an average of 70% of treated cows showing oestrus and ovulation within five days of treatment removal. This result however was significantly improved by replacing the equine chorionic gonadotrophin at time of device removal with 1 mg of oestradiol benzoate delivered by injection 24 hours after device removal.

The present invention is however directed to a more controlled formulation release as and when required, without the need to administer separate treatments. It provides autonomous delivery of the actives at a single site and to a predetermined delivery regime. It also relies on specific formulations developed for use with a unique, single application, breeding device designed to release an effective profile of hormones. Further, it is directed to achieving progesterone release maintained at a constant rate throughout treatment rather than the gradual decline in release experienced with passive release type devices. This is to overcome any possible affect of lower serum progesterone concentrations on fertility. The delivery regime further effects blood serum levels for oestradiol for only the period of time required to effect the desired outcome, rather than maintaining higher blood serum levels than required for prolonged periods (such as 24 hours) as has been evident in the prior art. In addition, it relies on the use of significantly greater ratios of actives:carriers than evident previously in the prior art, as discussed previously.

To improve shelf-life, application, and transportation of the formulation, various trials have indicated that powdered or tabletised actives are preferred. However, use of the actives in liquid form is acceptable in some embodiments. In such situations the inclusion of a preservative aids the shelf life of the liquid forms of the formulations. Such preservatives include magnesium stearate, Unigerm™. However, of particular importance is the use of water soluble preservatives that are recognised to be of injectable quality—as required to meet miinimum standards. Various other excipients may be added to the formulations as required. Such as free-flowing agents in dry formulations, binders, colourants, and so forth. Such excipients, their properties and typical quantities used, will be known to those skilled in the art.

The device used to deliver the formulations into the animal is located in the anterior vagina of the cow and delivers the actives to the vaginal mucosa preferably through a unique pressure/pumping delivery system. The preferred oestradiol and prostaglandin formulations are preferably delivered as single doses through a pot release and/or an automated syringe mechanism. There being two such pot releases of the oestradiol and one of prostaglandin, as required to effect the desired hormone regime to effect synchrony and oestrus in the target animal, which in the discussion above has related specifically to cows. The progesterone formulation is delivered from a collapsible bellows reservoir.

The device is retained for the purpose of controlling oestrus in the preferred embodiments, by appropriate retention apparatus associated with the delivery device. Further discussion, in brief, about the delivery device follows.

### BRIEF DESCRIPTION OF DIAGRAMS

Further aspects of the present invention will become apparent from the following description given by way of example only and with reference to the accompanying diagrams in which:

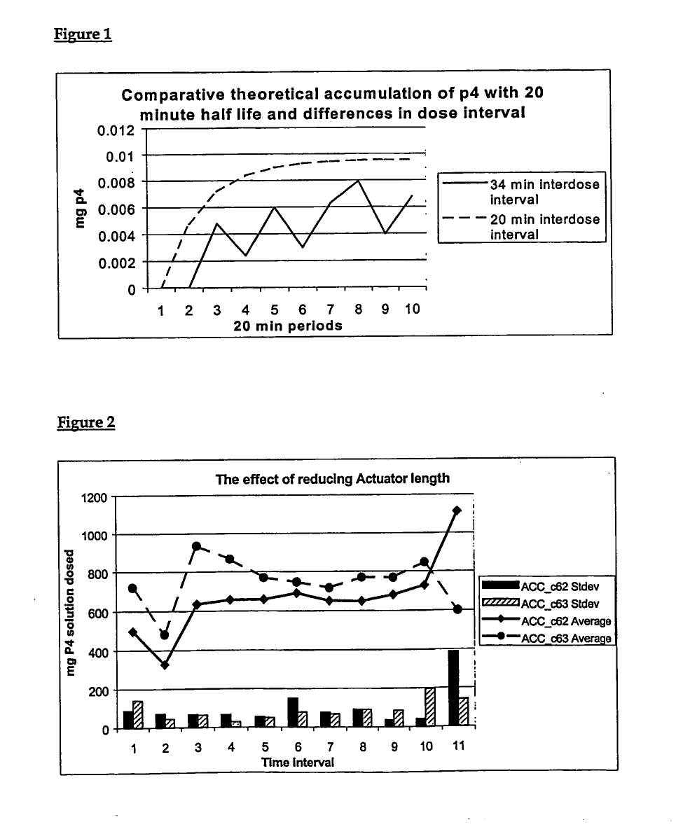

FIG. 1 is a graph showing the theoretical quantity of progesterone available with differing dose intervals from trials to match the output of the formulated hormone carrier solution to the specifications of the preferred intravaginal delivery system for use with cows in accordance with one preferred embodiment of the present invention, and

FIG. 2 is a graph showing the effect (from calibration trials) of a change in one component where all other specifications of the preferred intravaginal delivery system remain the same in accordance with one preferred embodiment of the present invention, and

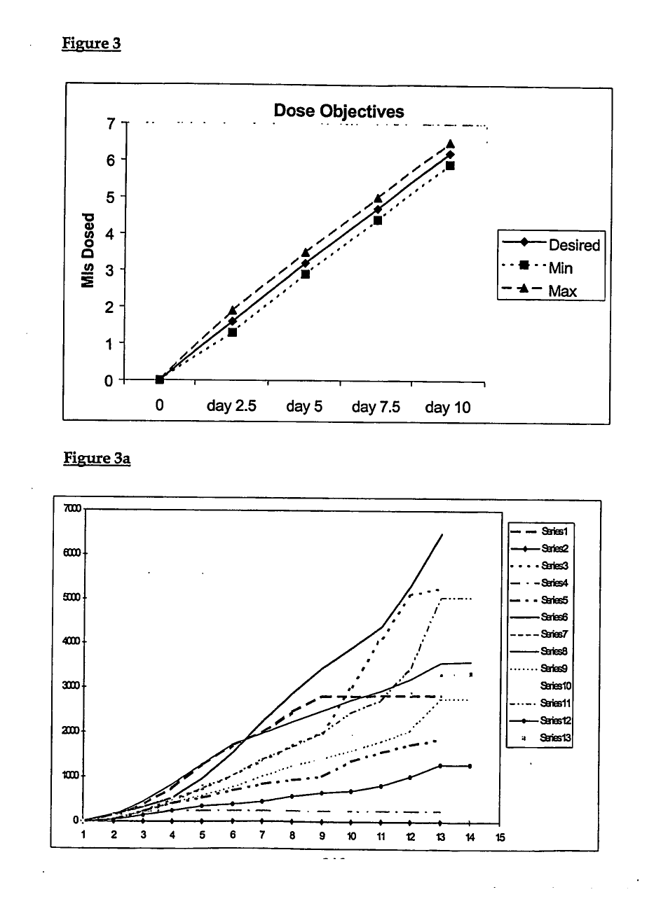

FIG. 3 is a graph showing dose objectives for in vitro calibration trials to match the output of the formulated hormone carrier solution to the specifications of the preferred intravaginal delivery system for use with cows in accordance with one preferred embodiment of the present invention, and

FIG. 3a is a graph showing a series of in vitro recalibration trials, to tune output of progesterone solution to match the delivery device specification in accordance with one preferred embodiment of the present invention, and

FIG. 4 is a graph identifying the accelerated dose output result and the dose output achieved in vivo for preferred formulations in accordance with one preferred embodiment of the present invention, and shows results for the completion of final calibration work of FIG. 3a, and

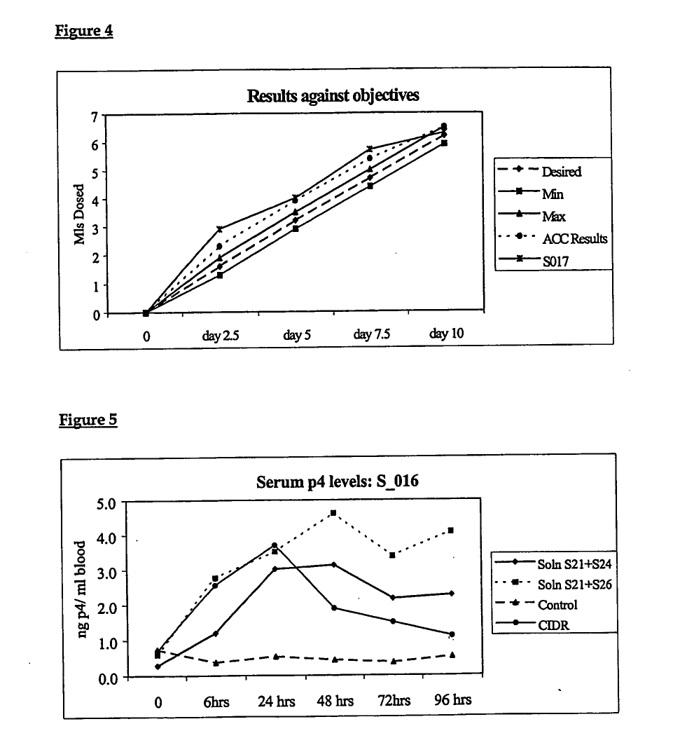

FIG. 5 is a graph illustrating maintenance of animal p4 blood serum levels using test formulations containing the preferred hormones and carrier solutions in accordance with preferred embodiments of the present invention compared with results obtained using a control and solutions released from an alternate progesterone delivery device; and

FIGS. 6-8 are graphs illustrating performance of the delivery device as calibrated to the preferred specification for pulsatile dosing of progesterone in accordance with one preferred embodiment of the present invention; and

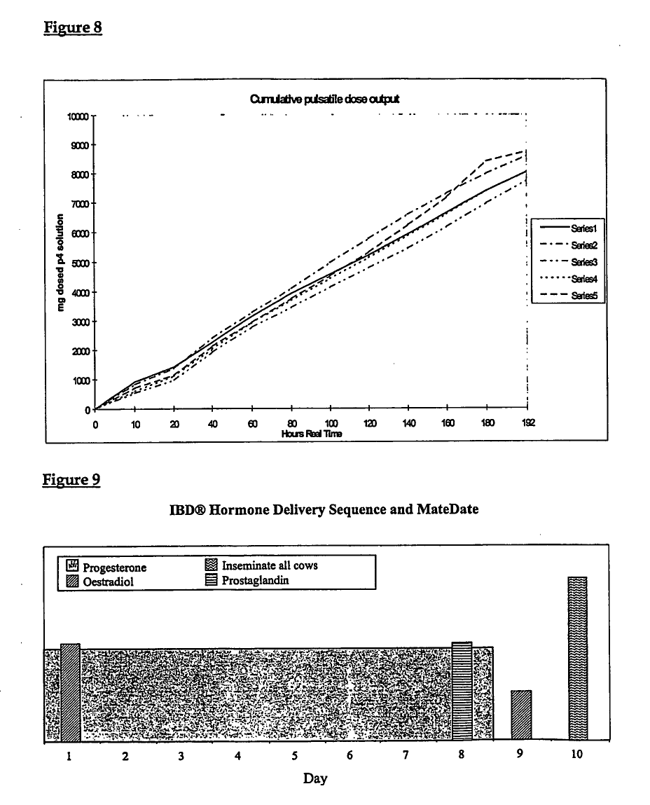

FIG. 9 is a graph illustrating the delivery regime for an 8-day programme for preferred formulations in accordance with one preferred embodiment of the present invention, and

FIG. 10 is a graph illustrating peak animal blood serum levels using test formulations containing the preferred oestradiol hormone dose and carrier in accordance with preferred embodiments of the present invention; and

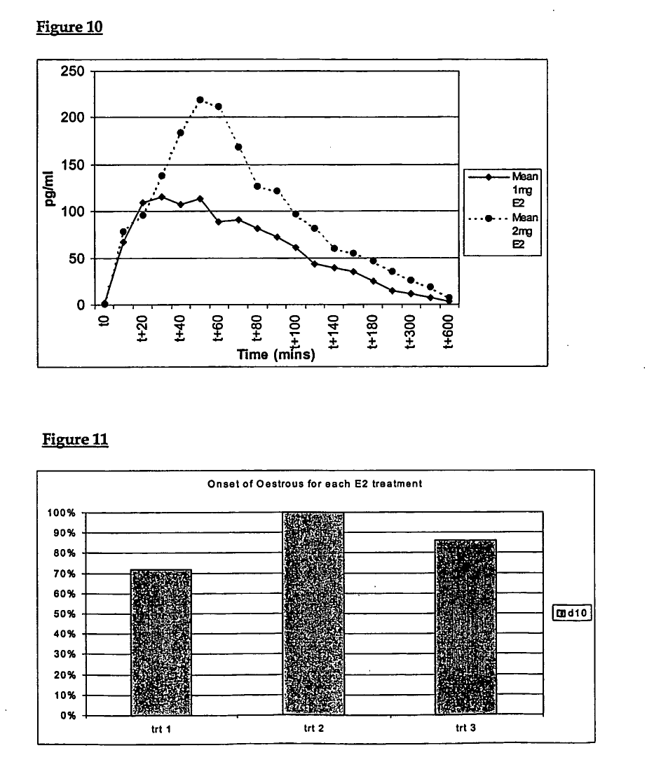

FIG. 11 is a graph showing synchrony of oestrus in test animals in trials of an 8-day treatment programme using the formulations in accordance with another preferred embodiment of the present invention.

### BEST MODES FOR CARRYING OUT THE INVENTION

The invention is now further described by way of examples with reference made to the above mentioned figures. However, it should be appreciated the various examples describe aspects of the invention as it relates to the control of oestrus and that the invention may have application to controlling other biological functions. The term “biological function” as used in this specification, includes any function, process or activity in an animal, capable of being artificially controlled by the use of chemical actives intervention of particular formulations, introduced under a specific regime and via a specific delivery device, within the scope of this invention. While reproductive capability (oestrus) is one example, other examples include, but are not limited to, growth, control of parasites, nutritiondigestive processes and so forth. Accordingly, variations will occur beyond these examples in relation to the potential formulations (number and type of actives and/or carriers, concentrations, form), the delivery regimes, configuration of the delivery device, location of the delivery device and by extension the single delivery site, and so forth.

### EXAMPLE 1

### The Administering Device

The formulations of this invention are used with controlled substance delivery devices. In the present example, these are controlled breeding devices used to synchronise the oestrous cycle in cattle for fixed time blanket insemination in either cycling or non-cycling cows. This is achieved by the accurate delivery of a complex hormone regime through a preferred system involving control (preferably electronic computer control) of a unique pumping system.

The preferred device ensures accurate delivery of 4 different hormonal formulations to the animal at precisely the required dose and at the exact time during an appropriate “x”-day treatment period. For example, a delivery period of 9-days to 11-days may be contained within a total treatment period from 10 days to 12 days, respectively. The length of the treatment periods and the format of the delivery regimes will, at least in part, be dictated by the requirements of the particular species. Different species may require differently programmed delivery regimes and different treatment lengths.

The ensuing description relates to the formulations (including preferred hormones and preferred carried solutions) and the delivery device for use in synchronising oestrus in cattle. However, it should be appreciated this invention may have application in the delivery of a range of formulations (via appropriately adapted devices) into other areas of the body to control or synchronise other body functions/cycles.

With reference to this present embodiment of the invention, the administering device is an intravaginal delivery device, developed to deliver the required hormones in required doses at required times into the anterior vagina of the animal for which synchronised oestrus is required.

The device is adapted to be retained in the animal in the preferred delivery site for at least the duration of the delivery regime. The method of retention may vary depending on the physiological site requirements in the animal, the behavioural characteristics of the animal for which control of the biological function is being effected, and the physical structure of the delivery device itself. The retention system may therefore be externally applied to the animal and be attached to the delivery device located internally of the animal; or be internal structures on or associated with an internally located delivery device; or involve an external delivery device having external retention apparatus, but with delivery conduits inserted into the animal. Other combinations will be apparent to those skilled in the art.

The intravaginal device is preferably made of pharmacologically safe materials, for use in animals, that also do not react with the formulation compounds and solutions being administered by the device. Plastic materials are preferably used. Plastic materials included in and on one embodiment of the device, that are exposed to contact with the treated animal are listed as follows:

| | |
|---|---|
| Device body | polypropylene copolymer |
| Tail | polyurethane elastomer |
| Chassis and nose | polypropylene |

All plastic materials exposed to the animal are in compliance with FDA regulation 21CFR 177.2600 and are suitable for food contact grade applications.

Whilst the administering device in the described embodiment of this invention is an intravaginal device, it should be appreciated that the administering device may be specific to the area of the animal's body into which a preferred formulation is to be administered—for example, an intraruminal device.

One preferred design concept for continuous/pulsatile dosing has the carrier solution containing one of the hormone actives retained within a collapsible reservoir. The reservoir is filled so it is devoid of air and is maintained under positive pressure by means of a force applied to the back end. Due to the positive pressure, fluid is always presented to the inlet of the micropump to which the reservoir is attached regardless of the attitude of the device. The control of the delivery from the reservoir is effected by electronic control means. However, a range of structural features of the delivery apparatus and/or changes to the control software also affect delivery. Such structural features include spring tension, compression of the collapsible reservoir, and so forth. Changes in delivery output particularly due to changes in the actuator length of the delivery apparatus, are discussed in with reference to the figures and tables referred to in Example 2.

Another preferred concept for continuous dosing has the carrier solution stored separately from the hormone active until prior to release of the formulation into the animal, when the carrier solution is introduced to the hormone active. The dissolved final formulation is then released. Yet a further embodiment may include the carrier being released in the vicinity of the separately delivered hormone, with mixing occurring in the anterior vagina. The hormone active may be available (for mixing with a carrier solution) in liquid, powdered or tablet form.

The preferred oestradiol and prostaglandin formulations are preferably delivered as single doses through a pot release and/or an automated plunger/syringe mechanism. There being two such pot releases of the oestradiol and one of prostaglandin, as required to effect the desired hormone regime to effect synchrony and oestrus in the target animal.

The specifics of the plunger/syringe dimensions, the number of unit doses that can be delivered and the timing of delivery are all relevant to the requirements of the product. In other words, more than one unit dose can be delivered or as many unit, doses can be delivered as is physically possible.

### EXAMPLE 2

### Preferred Actives

Reference is made throughout this specification to the use of hormone formulations used to control oestrus in cattle. However, it should be appreciated that an understanding of the concepts of the delivery regime, the single site delivery, the delivery apparatus, the controlled delivery, the method of delivery and actives:carrier ratios can be adapted as required for the use of other preparations/formulations and applications depending on the body process/cycle required to be controlled/synchronised.

As mentioned previously, in one embodiment described, some if not all of the hormones are preferably dissolved in a solution for efficacy of dosing and to ensure optimum transmucosal transfer. Alternatively, other embodiments may include a one or a combination of the different possible physical forms of the active hormones, which may also be tabletised, be micronised powders, be in gaseous form, or whatever form may be required to effect the desired delivery. All hormonal formulations used for this invention are put through stability trials and microbial tests. Quality control issues and microbial tests are discussed in a later example.

In one embodiment of the present invention the administering/dosing device preferably releases formulations of progesterone, an oestradiol (oestradiol benzoate or oestradiol 17β) and a prostaglandin (cloprostenol sodium) and uses any one or more of alcohols, fatty acids, cyclodextrins as the solvent/carrier. One described embodiment discusses the use of benzyl alcohol as a carrier solvent. Embodiments using other preferred solvents to benzyl alcohol as carrier solutions are described later. Other embodiments where the formulations may include use of progesterone and oestradiol 17-β and prostaglandin with preferred carrier(s) in tablet form are also described later.

The following discussion relates to each of the hormone actives used in the present invention and appropriate carriers in accordance with one embodiment as it relates to the example of controlling oestrus within the ambit of the present invention.

A) Progesterone

Progesterone occurs naturally in mammals and is normally present in dairy products and tissues of untreated animals. Progesterone is absorbed when administered vaginally, rectally, buccally, nasally and is rapidly absorbed from the site of an oily intramuscular injection. It is not well absorbed through ingestion, as it is partly destroyed in the liver. The half-life of progesterone in blood is only a few minutes. Progesterone is metabolised in the liver where about 12% is converted into a reduction product, pregnanediol, which is excreted in the urine conjugated with glucuronic acid.

The FAO/WHO publication on food residues have considered it unnecessary to establish an acceptable human daily intake or acceptable residual levels in food for endogenous hormones such as progesterone, as their presence is small in comparison to daily human production rates.

The quantities of progesterone in the formulation released from the preferred breeding device produce serum progesterone levels which compare to the serum progesterone levels produced endogenously from a normally functioning corpus luteum in a cycling cow. This is much less than that found in a cow in mid to late pregnancy. It is apparent from this comparison that tissue residues caused by release of the hormone in the formulation of the present application will be insignificant when compared to levels produced by normally pregnant animals.

As a natural steroid hormone secreted by the luteal cells of the corpus luteum and also by the placenta during pregnancy, progesterone is necessary for the preparation of the uterus for implantation of the fertilised oocyte and for maintenance of pregnancy.

The progesterone active and carrier formulation in one preferred embodiment of this invention is delivered as a continuous series of pulsatile doses.

One embodiment of the intravaginal breeding device used to administer the formulation to the animal is configured to hold at least 2 grams of natural powdered progesterone. In addition to the progesterone, the intravaginal device also contains preferably 3 mls of benzyl alcohol, which weighs approximately 3263 grams. This gives a total weight of 5263 grams of solution, which equates to approximately 38% progesterone and 62% of benzyl alcohol in every 5 ml of the total solution.

In other embodiments the carrier solution is mixed with the progesterone in tablet form prior to release into the animal. Pre-mixed active:solutions may also be used.

The preferred dosage delivered in this embodiment from the intravaginal device, is 42 mg of progesterone solution every 2 hours. This equates to 5.040 grams of the total solution dosed over the treatment period. There will remain a small amount of solution in the valve and bellows of the intravaginal device unable to be dosed.

The daily dose rate during the treatment period is a maximum daily of approximately 200 mg/day via intravaginal administration. This is below the daily dose of most intravaginally administered products available on the New Zealand market, particularly in the early stages of the treatment period. This daily dose rate is at the lower end of the usual dosage range of 200-400 mg for horses or cattle, when administered by implantation into a non-edible part of the body. It should be appreciated however, that the quantity of progesterone, and carrier solution in a preferred dose regime may vary depending upon the species of animal to which the formulations are administered. Results demonstrated by administration to cattle are merely by way of an example of one application of the present invention.

Progesterone plasma concentrations (PPC) in mature, non-pregnant cows would not be expected to rise above the maximum normal physiological limits under this dose regime.

Using currently existing licenced products (i.e. Easibreed CIDR-B device produced by DEC International Limited in New Zealand), it was found that higher average progesterone plasma concentrations (PPC) were obtained using three such devices at the same time. For example, cows treated with three devices for 15 days showed average PPC of 8.4 ng/ml versus 2.8 ng/ml for cows treated with one device. Further, the difference between the initial and residual values of progesterone in the CIDR device after a 15 day treatment equated to 0.83 gms/device whether one or three devices per cow were inserted as the treatment This shows that the three CIDR's released a total average equivalent of 2.49 g over a fifteen-day treatment period and raises PPC's to an average of 8.4 ng/ml.

However, this is still well below the normal physiological levels of cows in late pregnancy, when the main source of progesterone formed is from the placenta For example, plasma progesterone concentrations in pregnant control animals from days 10 to 94 after ovulation showed over twice the concentrations found in non-bred animals treated with 150 mg progesterone/day.

Progesterone has an LD100 value in the rate of 327.1 mg/kg when administered interparenterally. Extrapolated, this information would suggest that the total release of all the contents of progesterone in the preferred administration device at one time would not indicate a toxic dose in mature cattle.

B) Prostaglandins

Prostaglandin delivered by the intravaginal route has been shown to have the same luteolytic effect as that when prostaglandin is administered intramuscularly.

Prostaglandin F_{2α} type is a prostaglandin that has a short half-life of three hours in the blood of treated cows. The preferred prostagladin used with formulations of the present invention, and the preferred intravaginal delivery device is cloprostenol sodium. This compound is an analogue of Dinoprost (prostaglandin F₂α). It is used as a luteolytic agent and acts on the smooth muscles. It induces contraction of uterine muscle at any stage of pregnancy and acts predominantly as a vasoconstrictor of blood vessels and bronchoconstrictor in bronchial muscle.

Whilst the prostaglandins PGF₂α have known side effects on smooth muscles and the central nervous system, cloprostenol is a product that has been licensed in New Zealand for 15 to 20 years and has been used extensively in the cattle breeding industry with no reported concerns over toxicity or residues. It is a product that is also licensed in Europe and in North America and is widely and safely used in both regions.

Tests directed at measuring residual concentrations of prostaglandin in tissues have shown that less than 0.75% of a dose of prostaglandin administered to a cow is eliminated in milk and this was largely contained in the samples taken 4 hours after administration. Detectable persistence of residues of prostaglandin in the edible tissues of the cow does not occur.

The amount of prostaglandin in the formulation of the present invention, as used in an intravaginal device of one embodiment of the present invention is preferably 240 μg (micrograms). Given the purity of the product according to the Certificate of Analysis is 96%, this will represent 240 micrograms of pure prostaglandin per unit dose.

The unit dose of 240 μg equates to an equivalent dose of less than 1 μg/kg when administered to a cow. No known side effects have been recorded in cows treated with the normal luteolytic dose of 500 μg and there are no reports of adverse reactions in humans for residues in animal product.

The formulation is released intravaginally from an intravaginal device as a dose of 240 μg in a spike release preferably at day 7.5 of treatment (note: during this stage of treatment the cow has not been mated). This is half of the recommended dose for therapeutic use in mating management for the currently licensed cloprostenol products available in the NZ market.

Further, the cloprostenol formulation in the intravaginal breeding device is used as a luteolytic agent in cycling cows. As cloprostenol has a short half-life, concentrations would return back to normal physiological levels before the cow is mated.

A three-year fertility study on seven hundred and thirty seven dairy cows reported no chronic side effects on subsequent fertility following cloprostenol induced luteolysis.

C) Oestradiol Benzoate

Oestradiol is a naturally occurring oestrogenic substance and is found in all mammals. All oestrogenic substances can be absorbed when taken orally through the gastro-intestinal tract and through skin and mucous membranes. It is partly bound (about 50%) to plasma proteins and is rapidly metabolised in the gut wall and the liver to the less active oestriol and oestrone. Some oestradiol undergoes enterohepatic recycling. Excretion occurs of the unchanged drug, sulphate and glucuronide esters in urine and a small amount in faeces.

A FAO/WHO publication on food residues considered it unnecessary to establish an acceptable daily intake or an acceptable residual level in food for endogenous hormones such a oestradiol. Appraisals on residues resulting from the use of oestradiol as a growth promoter in accordance with good animal husbandry, found that although the exogenous source of oestradiol raised mean tissue oestradiol concentrations, the amounts were very small when compared to daily production rates in humans and are unlikely to pose a hazard to human health.

Oestradiol benzoate is a semi-synthetic form of naturally occurring oestradiol. This is the preferred oestradiol used in the formulations of the present example by which this invention herein is described.

The intravaginal device used to administer the oestradiol formulation in one embodiment of the present invention is controlled to preferably deliver one spike release of the oestradiol benzoate (OBD). Release of the oestradiol occurs before the animal is mated. Release is on day one of treatment.

The dosage of oestradiol is well below the usual dose range for cattle of 50 mg as a single dose by intra-uterine infusion. There are also currently licensed products delivering 10 mg of oestradiol benzoate intravaginally with nil recommended withholding period.

The device delivers an initial dose of 7-8 mg of oestradiol by the intravaginal route. Doses of this magnitude fall within the physiological levels produced by the ovary during ovulation and have a very transient effect on tissue levels. For example, dosing non-pregnant heifers with 24 mg oestradiol controlled release implants showed oestrogen levels in tissues of slaughtered animals were not significantly different from untreated animals. More specifically the preferred amount of oestradiol benzoate in each intravaginal breeding device is 8 grams. With a purity of 97% this represents 7.7 grams of pure oestradiol.

In other embodiments of the intravaginal breeding device used to administer the formulations of the present invention into a cow, there is provision for administering two doses of oestradiol 17β as required to effect the preferred synchrony of oestrus. In this embodiment the doses are released from two independent reservoirs. Pot 1 preferably contains 6.8 g of the oestradiol and pot 3 contains 0.9 g of the oestradiol. In this embodiment, the formulations may be delivered in substantially solid form (as a tablet, capsule or micronised powder), or as a paste. Further details of the delivery regime for a two dose option are provided in example (D) below.

D) Examples of Programmes of Events

Table 1 illustrates administration of two doses of oestradiol as required to effect the preferred synchrony of oestrus as described in the discussion above. Table 2 and Table 3 illustrate two optional programmes of events in the administration of pulsatile doses of the progesterone active/carrier formulations from an intravaginal device as part of a programme to control oestrus in cows.

TABLE 1

| | Time | | | |
|---|---|---|---|---|
| Reading | (mins) | Event | Weigh P4 Son. | |
| Start | 0 | Start (dosing after 20 seconds) | 0 | grams |
| 1 | 10 | OBD 1 pot 1 fired (after | | |
| | | 40 secs) | | |
| 2 | 20 | | | |
| 3 | 30 | | Approx 2.5 | grms |
| 4 | 40 | PG pot 2 fired (after 36 mins) | | |
| 5 | 50 | | | |
| 6 | 60 | Dosing stopped (after | Approx 5 | grms |
| | | 59 mins) | | |
| 7 | 70 | OBD 2, pot 3 fired (after | | |
| | | 65 mins) | | |

TABLE 2

| V6.4 | Dose | Block | Block | Total Running Time | | |
|---|---|---|---|---|---|---|
| #doses | Interval | time (mins) | time (hrs) | mins | Hrs | Days |
| 10 | 0.017 | 0.2 | 0.00 | 0.2 | 0.0 | 0.00 |
| 30 | 20 | 600.0 | 10.00 | 600.2 | 10.0 | 0.42 |
| 62 | 34 | 2108.0 | 35.13 | 2708.2 | 45.1 | 1.88 |
| 61 | 34 | 2074.0 | 34.57 | 4782.2 | 79.7 | 3.32 |
| 80 | 30 | 2400.0 | 40.00 | 7182.2 | 119.7 | 4.99 |
| 48 | 25 | 1200.0 | 20.00 | 8382.2 | 139.7 | 5.82 |
| 150 | 20 | 3000.0 | 50.00 | 11382.2 | 189.7 | 7.90 |
| 50 | 0.017 | 0.8 | 0.01 | 11383.0 | 189.7 | 7.90 |
| 491 | | | | | | |

TABLE 3

| V6.4 | Dose | Block | Block | Total Running Time | | |
|---|---|---|---|---|---|---|
| #doses | Interval | time (mins) | time (hrs) | mins | Hrs | Days |
| 5 | 0.017 | 0.1 | 0.00 | 0.1 | 0.0 | 0.00 |
| 12 | 15 | 180.0 | 3.00 | 180.1 | 3.0 | 0.13 |
| 122 | 22 | 2684.0 | 44.73 | 2864.1 | 47.7 | 1.99 |
| 131 | 20 | 2620.0 | 43.67 | 5484.1 | 91.4 | 3.81 |
| 119 | 20 | 2380.0 | 39.67 | 7864.1 | 131.1 | 5.46 |
| 130 | 17 | 2210.0 | 36.83 | 10074.1 | 167.9 | 7.00 |
| 108 | 12 | 1296.0 | 21.60 | 11370.1 | 189.5 | 7.90 |
| 50 | 0.017 | 0.8 | 0.01 | 11370.9 | 189.5 | 7.90 |
| 677 | | | | | | |

Pot 1 burn 100 mins from switch on
Pot 2 burn 10820 mins from switch on
Pot 3 burn 12500 mins from switch on (1459 minutes after dose finish)

E) Dose Control Software

Dose control software for delivery of progesterone is programmed to deliver the active/carrier formulation as a continuous series of pulsatile doses.

The programme needs to accurately deliver a measured dose of active at interdose intervals of less than the metabolic rate of the active to have a high probability of maintaining serum values above a required threshold. Elevation and maintenance of progesterone serum levels to a minimum level of 2 ng/ml is preferred.

There is a very wide variation in the rate of metabolism between individual animals, which makes it very difficult to accurately determine the rate at which levels fall below the threshold. Dose titration studies using the current formulations in OVX (ovarectomised) cows, showed that serum levels for progesterone fall quickly in the interval between pulsatile doses.

A half-life of about 20 minutes was identified and the dosing profile altered accordingly. Long interdose intervals lead to ineffective maintenance of progesterone serum levels. The effect of this is best shown in FIG. 1 where the theoretical quantity of progesterone p4, available with differing dose intervals is shown for every 20-minute period.

For maintenance of serum levels above the threshold, dose control software needs to be able to compensate for the decreasing volume of solution delivered per dose over time. This is achieved by increasing the number of times the micropump operates during a given period. In addition to software development the mechanical components of the delivery device have been developed to fully understand the effect of deviation from specification of various device configurations. Even small changes can have dramatic effects on dose variability and total output.

FIG. 2, from calibration trials, shows the effect of change with one component of the delivery apparatus of the delivery device, where all other specifications remain the same. In this example the actuator length was reduced by 0.125 mm in ACC_c62. This is a practical example of the effect of changing the stroke of the pump. Correct calibration is vital to optimise dose performance of the pulsatile delivery, minimise dose variance and develop a set of component specifications that are robust and commercially manufacturable.

F) Other Possible Active Compounds

In the present invention directed at synchronising oestrus in cattle, the active ingredients are released into the vagina from the dosing device and are carried through the vaginal mucosa by a preferred carrier. The vaginal mucosa is a soft tissue lining with fine capillaries that provides an ideal access site for absorption of the actives.

The objective is to raise the blood serum of the animal by having the active ingredient become readily absorbed and metabolised. Dissolving the activities particularly where the actives are sparingly insoluble) in an appropriate carrier solution allows the active ingredient to be readily spread over the mucosa surface, and consequently absorbed.

Whilst oestradiol benzoate, progesterone and cloprostenol sodium are the preferred active compounds for use with the intravaginal breeding device in controlling/synchronising oestrus cycling, other “active” compounds may also be used. The opportunity for using alternate hormones such as gonadotrophin hormone (GNrH) in combination with some of the existing hormones currently used with the intravaginal breeding device, has been identified for example.

Further, there are a number of possible variations on the quantity of the active ingredients currently used in the breeding device that could be delivered. This could be achieved through either variation of the total quantity available to be delivered or alternatively by changes to the dose software enabling a different regime to be established during use. The possibility of other suitable “actives” is also particularly relevant where the device administers compounds in vivo in other body cavities and for other purposes.

### EXAMPLE 3

The range of possible concentrations of the “hormone actives” in the solutions as a preferred range is discussed below, as is the procedure used to determine the concentration and stability of the respective hormonal solutions used in the formulations (in accordance with one embodiment of the present invention as described with reference to controlling oestrus—particularly in cattle). The procedure for analysing oestradiol and prostaglandin concentration levels uses high-pressure liquid chromatography. The procedure for determining progesterone concentrations uses UV Spectrophotometer according to the British Pharmacopoeia 1993 Standards.

A) Procedure and Calculations for Determining Preferred Concentration of Oestradiol Benzoate, Cloprostenol Sodium, Progesterone in Formulation.

Oestradiol

The concentration of the oestradiol ingredient is calculated as follows: A graph of standard concentration versus peak area is drawn, which will determine the concentration of oestradiol in the sample solution.

$$1.75 \% w \text{/} w Oestradiol solution$$

$$\% w \text{/} w = mg \text{/} ml ( graph ) \times \frac{100 \times 100}{1000 \times SG}$$

$$0.25 \% w \text{/} w Oestradiol solution$$

$$\% w \text{/} w = mg \text{/} ml ( graph ) \times \frac{50 \times 100}{2 \times 1000 \times SG}$$

Prostaglandin

A graph of standard concentration versus peak area is drawn which determines the concentration of Sodium cloprostenol in the sample solution.

$$\% w \text{/} w = mg \text{/} ml ( graph ) \times \frac{50 \times 100}{5 \times 1000 \times SG}$$

Progesterone

$$\% P 4 w \text{/} w = \frac{absorbency of sample \times 50 \times 100 \times 250 \times 100}{535 \times 20 \times 20 \times 100 \times sample weight}$$

### EXAMPLE 4

### Carrier Solutions

A carrier for the purpose of this specification is any means in solid or fluid form (such as powder, tablet, liquid, paste, gas, and so forth) that assists the transfer, transport, absorption, solubility (and so forth) of an active (such as a chemical, hormone and so forth) across a physiological barrier (such as a membrane), to effect improved bioavailabiliy of the active to the animal (such as increased levels in the blood) and so forth.

Benzyl Alcohol

Benzyl alcohol was originally chosen as one preferred carrier of the hormone active (particularly progesterone) in the preferred intravaginal delivery device. Benzyl alcohol has the ability to be saturated with progesterone to high levels of 38%-40% w/v, in a stable solution, without precipitation during storage or operation. Other benefits associated with its use as a carrier are:
a) It does not accumulate.
b) It is readily absorbed from the gastrointestinal tract and rapidly broken down to benzoic acid that is metabolised and excreted as hippuric acid in the urine. For example, within 6 hours of ingesting 1.5 g of benzyl alcohol human subjects eliminated 75% to 85% of the dose in the urine as hippuric acid.
c) It operates well as the carrier of all the actives used in the delivery device to deliver hormones through the vaginal mucosa of the cow.
d) It is normally stable, but does however oxidise to benzaldehyde and benzoic acid when exposed to the air.

However, for long term efficacy of the intravaginal breeding device in overseas markets, benzyl alcohol has been identified as a less suitable solvent due to problems associated with transportation and potential regulatory and FDA issues.

Nevertheless, where the carrier solution is benzyl alcohol, the method by which the solutions are mixed and/or released into the animal from the intravaginal device is described below.

For one of the embodiments directed to the effective control of oestrus in lactating cows, four different drug formulations (prostaglandin, oestradiol, oestradiol and progesterone) are required.

These are provided in Table 4 below:

TABLE 4

| Formulation | Wgt | | | | | | |
|---|---|---|---|---|---|---|---|
| Components | (% TotWgt) | | | Form different from (1) | | | |
| Prostaglandin | 400 | mg | (0.38) | Benzyl Alcohol | 399.75 | mg | 99.94% |
| | | | | Cloprostenol sodium | 0.25 | mg | 0.06% |
| Oestradiol (1 mg) | 400 | mg | (0.38) | Benzyl Alcohol | 393 | mg | 98.25% |
| | 400 | mg | (0.38) | Benzyl Alcohol | 399 | mg | 99.75% |
| Oestradial (1 mg) | 400 | mg | (0.38) | Benzyl Alcohol | 399 | mg | 99.75% |
| | | | | Oestrodiol | 1 | mg | 0.25% |
| Progesterone | 5263.16 | mg | (4.93) | Benzyl Alcohol | 3263.16 | mg | 62% |
| | | | | Progesterone | 2000 | mg | 38% |

TABLE 5
Solvents and Solutions used in Trials

| Solvent/solution mix with progesterone | Percent |
|---|---|
| Polyethylene glycol ether P2000 | 100% |
| Marlophen NP5 | 100% |
| Dioxane + Marlophen NP5 | 28-60% NP5 |
| 2-phenylethanol | 100% |
| 2-phenylethanol + Marlophen NP3 | 2.5-10% NP3 |
| 2-phenylethanol + Marlophen NP5 | 2.5-10% NP5 |
| 2-phenylethanol + Marlophen NP9.5 | 2.5-10% NP9.5 |
| 2-phenylethanol + Polypropylene glycol P1000 | 2.5-10% PPG |
| 3-phenyl-1-propanol | 100% |
| 3-phenyl-1-propanol + Marlophen NP3 | 2.5-10% NP3 |
| 3-phenyl-1-propanol + Marlophen NP5 | 2.5-10% NP5 |
| 3-phenyl-1-propanol + Marlophen NP9.5 | 2.5-10% NP9.5 |
| 3-phenyl-1-propanol + Polypropylene glycol P1000 | 2.5-10% PPG |
| DL-1-Phenylethanol | 100% |
| DL-1-Phylethanol + Marlophen NP3 | 2.5% NP3 |

Alternative Carrier Solution(s)

Whilst benzyl alcohol has been used as the solvent and carrier of the active because of its ability to contain a high concentration of active in solution and to transfer the active across the vaginal mucosa, problems associated with the use of benzyl alcohol led to an investigation of alternative proprietary solutions, including distilled water, for dispensing the hormone actives (particularly progesterone) in solution from an in vivo delivery device.

Any alternate solution needed to be capable of dissolving 2 grams of micronised progesterone powder and also remain stable, be non-flammable, non-toxic and allow release of progesterone in vivo to meet the goal of elevating progesterone (p4) blood serum levels (levels in serum 4 days after administration of in vivo progesterone) beyond a threshold level of 2 ng/ml.

The solution also had to be delivered accurately from the preferred delivery device to meet delivery specifications and be non-traumatic in all respects to the animal. It also needed to be compatible with materials used in the delivery device. Accordingly, other suitable carrier solutions, mixed with the preferred active ingredients were trailed.

The results of the solvents and solutions used in trials are listed in Table 5 above.

Summary of Results

A number of base solvents were screened that met the objectives. The most likely solvents were tested and assessed for their ability to hold 2 grams of progesterone within 7 mls of solution. The solvents were required to remain stable with no signs of precipitation at extreme temperatures (5°-40° C.) likely to be encountered by the delivery devices used, in particular intravaginal delivery devices. From this primary screening, two solutions met all criteria and remained stable. Two base solvents were identified that maintained stability with 2 grams of progesterone in a 7 ml solution. A surfactant was also identified that allowed the solution to be thickened.

A pilot study of 20 cows demonstrated elevation of serum p4 levels (levels of progesterone four days after administration of the formulation) with the two preferred formulations. These formulations included phenylethanol in the carrier solution.

The polyethylene glycol and phenylethanol polymer chemistry group was then investigated in more detail. Further trials were conducted to check the solubility over time and specific environmental conditions of a solution containing 2 gm of micronised progesterone powder. Samples were tested at 4° C. and 37° C. for stability. Further, initial screening required that solutions had to remain stable for a minimum period of 10 days. The results provided in FIG. 3 relate to a 10-day optional programme:

FIG. 3a illustrates a series of in vitro recalibration trials, to tune output of the progesterone solution to match the delivery device specification in accordance with one preferred embodiment of the present invention.

In excess of 100 delivery devices were run in in vitro calibration trials to tune output of progesterone solution to match the delivery device specifications.

The new formulation(s) enabled the delivery device (such as the previously described intravaginal breeding device for cows) to be used without the need to make major design changes to components of the device. The formulation(s) did not cause irritation in the cow or create large amounts of mucous.

The dose results achieved from this in vitro work were reliable yet possibly provided a higher-than-desired output. It became important at this stage to ascertain:
a) The correlation between actual in vivo results and the accelerated in vitro calibration results; and
b) The efficacy of the new solutions at raising blood serum p4 levels.

FIG. 4 identifies the dose correlation between the accelerated (ACC) dose output result and the dose output achieved in vivo. Based on this correlation information, it was possible to accurately determine the relationship between ACC and in vivo results for the completion of final calibration work. Trials relating to this aspect of the invention involved 41 lactating dairy cows.

The pilot study previously discussed using 20 cows was conducted with two solutions. Solution 1 was a combination of phenylethanol plus Marlophen NP3 plus 2 gm progesterone and Solution 2 was a combination of 2-phenylethanol plus polypropylene glycol P1000 plus 2 gm progesterone. These solutions were trialed in previously synchronised animals to ascertain that p4 blood serum levels could be elevated using the new solutions.

FIG. 5 illustrates the results (again for a 10-day optional programme) from these two solutions and compares results with an alternate progesterone delivery device available (the CIDR device). All animals treated with the new solutions in this trial, administered using the preferred delivery device, maintained blood serum level above the threshold of 2 ng. A control group using the delivery device to deliver a control solution (no progesterone) was also monitored and is featured in this graph.

From this pilot trial both solutions demonstrated their ability to satisfactorily elevate progesterone blood serum levels above the target threshold level for 96 hours. The supervising veterinarian in this trial recorded no irritation, adverse mucous development or animal trauma.

Alternate to Benzyl Alcohol Status Summary

a) Benzyl alcohol raised issues of material compatibility and potential fire risk.
b) The decision was made to evaluate alternate solutions that met specified criteria.
c) Two options identified had to meet initial in vitro and in vivo criteria.
d) Re-calibration of an existing preferred delivery device was required due to significantly different viscosity characteristics.
e) Solution 1 was unlikely to meet regulatory criteria in the US.
f) Calibration and efficacy trials with were continued with Solution 2. In vivo trials were done in OVX (ovarectomised) and entire cows to confirm efficacy.
g) No specific confirmation of material compatibility with new solutions has been conducted. However, preliminary considerations confirm compatibility with all the materials of the preferred delivery device.

### EXAMPLE 5

### Mixing Formulations

It should be appreciated that the following description relates to one example of the steps involved in mixing the formulations, with particular reference to benzyl alcohol as the carrier. Depending on the active and the carrier used the processes and ingredients will vary.

A) Using Benzyl Alcohol as the Carrier

As progesterone, prostaglandin and oestradiol readily dissolve in benzyl alcohol, mixing of the formulations simply requires the addition of the required ingredients to a mixing tank with steady mixing to complete dissolution (120 minutes), taking care to prevent cross contamination of the active. After mixing, the respective tanks are sealed and washed with isopropyl alcohol.

Method for Progesterone

a) Measure the required amount of benzyl alcohol to achieve the correct % w:w into a clean and dry 20 litre stainless steel tank. This is sufficient for 4000×5 ml doses.
b) Weigh the correct amount of progesterone into a separate clean and dry container.
c) Slowly and carefully add the progesterone to the benzyl alcohol.
d) Attach the lid ensuring it is firmly closed. Activate the mixer, mixing slowly for a least 2 hours or until completely dissolved.
e) At the completion of the mixing process decant into a clearly identified storage contained until required. Thoroughly clean all mixing utensils in Isopropyl, disposing of washings by incinerating in a furnace.
f) Ensure the benzyl alcohol to be used in the solutions has passed through a 10μ filter prior to mixing.
   Method for Oestradiol Benzoate

There are preferably two different concentrations of oestradiol solutions used in the intravaginal breeding device administering these formulations into cows.
a) Measure the required amount of benzyl alcohol to achieve the correct % w/w into a clean and dry 1 litre stainless steel container. This is sufficient for 2500×0.4 ml doses.
b) Weigh the correct amount of oestradiol into a separate clean and dry container.
c) Slowly and carefully add the oestradiol to the benzyl alcohol.
d) Thoroughly mix until dissolved.
   Method for Prostaglandin
a) Measure the required amount of benzyl alcohol to achieve the correct % w/w into a clean and dry 1 litre stainless steel container. This is sufficient for 2500×0.4 ml doses.
b) Weigh the correct amount of the preferred prostaglandin into a separate clean and dry container.
c) Slowly and carefully add the prostaglandin to the benzyl alcohol.
d) Thoroughly mix until dissolved.

As all of the hormonal solutions are potentially dangerous (especially in their raw (powder) state), protective clothing is worn whilst handling the products.

### EXAMPLE 6

A) Unit Dose Formulations

Within the intravaginal delivery device used, some of the actives are contained within pots located in the front section of the device. These actives are typically delivered as unit doses. The progesterone active is typically contained in a progesterone reservoir.

In embodiments using benzyl alcohol as a solvent for the actives, the characteristics of this solvent became particularly relevant. Benzyl alcohol is a product with very low surface tension with the ability to creep along the finest of cracks or gap. Consequently, in the design of the delivery device, seals had to maintain the highest integrity with this solution to prevent any leakage.

Further, benzyl alcohol was identified as particularly unsuitable as a carrier of the unit doses due to problems associated with transportation (due to air expansion and contraction which is inevitable during transit of delivery devices containing fluids particularly via air transport with associated pressure differentials in a plane's cargo hold) and potential regulatory FDA issues. It was therefore preferable that an alternative carrier to benzyl alcohol be found.

However, even using alternative carrier solutions issues of expansion and contraction during air transportation remained. Accordingly, it was elected that a change of formulation particularly of the two oestradiol and one prostaglandin actives from a liquid to a capsule or tablet be evaluated. Prostaglandin was considered to be readily available to the animal in this form via the vaginal mucosa and previously, intravaginal oestradiol capsules of varying concentrations had proven efficacy.

Removing liquids from the front pots of the delivery device and replacing with encapsulated actives prevented transit spillage yet still allowed acceptable “expansion and contraction” of the components of the delivery device during altitude variance without compromising seal integrity. Suitable packaging also assisted this process.

Development focussed firstly on in vitro and in vivo trials conducted to confirm the delivery device in its current configuration was capable of delivering placebo tablets with a high degree of reliability. A pilot study confirmed the design of the nose pots were capable of:
a) Housing a tablet.
b) Releasing the contents to the cow upon the plunger firing as instructed by the control (accelerated software) program of the device.
c) Retaining the tablets within the nose pots under typical air pressure fluctuations likely to be experienced in air transit.

Sixty devices were subjected to pressure increases to reach a maximum 15″ Hg and held at that pressure for 10 minutes in a vacuum vessel. A hundred percent of the devices retained their tablets (N=1080) as shown in Table 6.

TABLE 6
FMEA summary of pot functions in Solution and efficacy trials
indicating improved delivery of the tablets
IBD ®'s recovered from 360 cows

| | | |
|---|---|---|
| Pot fire | 1 | 100% |
| (N = 1080) | 2 | 100% |
| | 3 | 100% |
| Contents of the pots been expelled | 1 | 100% |
| (N = 1080) | 2 | 100% |
| | 3 | 100% |

Some slight movement of the nose cap was noted and the recommendation to develop a shipping cap or restraint as part of the delivery device packaging was identified.

A cow trial was designed to verify the delivery device's effectively delivery of the unit doses in tablet form over a 12-day treatment. The objective of the trial was to determine if the existing unit dose mechanism in the device would effectively operate with a “pill” as the carrier, in vivo. A placebo pill was “designed” by hand-shaping a confectionery item to fit the existing delivery device's active containing chamber.

The Summary of Results is:

a) 91% (104) of the tablets were expelled.
b) 9% (10) tablets were not expelled. This failure was attributed to the test “tablets” sticking in the chamber as a result of an inability to uniformly configure the prototype “pills”.

The results demonstrated device capability, in most cases, to deliver the active in solid form to the animal. Some product modification, principally to the plunger/pressure means of the delivery apparatus of the delivery device was required to ensure the pills were presented to the cow rather than relying on moisture to dissolve the active within the nose pot.

After confirmation of the delivery device capability to deliver solid capsules from the three front pots, the active ingredients were encapsulated within a quick dissolving tablet. The three front pots of the delivery device would therefore deliver their active ingredients via three tablets, delivered according to the product specifications (discussed in example 8 below).

B) Unit Dose Tablets

The doses of oestradiol and prostaglandin contained within the three single dose chambers were contained within a solid capsule. The tablets produced have the following description:
a) Round, coloured (according to active), biconvex tablets, plain on both sides
b) Average thickness: 3.18 mm
c) Average diameter: 4.78 mm
d) Disintegration in water: 16 seconds.
e) Hardness: Average 6.7 scu
f) Average weight: 60.7 mg
g) The oestradiol (1) tablet contains 7 mg of Oestradiol Benzoate
h) The prostaglandin dose is 240 mcg of Sodium Chloprostenol
i) The oestradiol (2) tablet contains 2 mg of Oestradiol Benzoate

Whilst the above characteristics are preferred for one embodiment of the present invention, it should be appreciated the configuration of the “pills/tablets” may vary depending on the actives used, quantity required, region of the animal's body into which the tablet is released and the specifications of the delivery device adapted to administer the dose. For example, the oestradiol tablets may contain a range from 1 mg to 10 mg of oestradiol benzoate, and so forth.

The preferred solvents for use with the progesterone of this embodiment of the present invention are Marlophen NP3 and/or Propylene glycol P1000 and/or 2-Phenylethanol.

C) Pulsatile Dosing

Progesterone is delivered in solution as a continuous programmed series of pulsatile doses delivered in accordance with the preferred delivery regime and in response to commands from the programmable electronic control means of the delivery device. The design concept for continuous dosing has the solution containing progesterone retained within a collapsible reservoir. The reservoir is filled so it is devoid of air and is maintained under positive pressure by means of a spring force applied to the back end. Due to the positive pressure, fluid is always presented to the inlet of the micropump to which the reservoir is attached regardless of the attitude of the device.

The micropump is operated by means of a magnetic core that is forced to stroke when the electromagnetic coil within which it is housed is energised. The core activates a release mechanism that allows a predetermined quantity of solution to enter the micropump. The positive pressure in the reservoir ensures solution enters the micropump and is expelled from the outlet under pressure. The micropump is very energy efficient to enable small, low energy, power cells to be used.

Many biological functions are based on hormonal interactions involving the integrated responses of a number of substances as in the case of the oestrus cycle. When attempting to control a biological function the delivery of the actives to effect their interaction and hence achieve the desired responses is one area where controls may be implemented.

The preferred delivery device is effective in controlling the time of delivery of a substance, as well as the volume delivered on a dose by dose basis. The control of delivery is gained through specialist dose control software (as discussed previously) that is programmed into a microchip. The microchip is energised by a miniature power cell and controls operation through a printed circuit board and electromagnetic coil.

The delivery regime can follow any sequence depending on the dose control software programmed into the microchip. With the preferred delivery device and its application in controlling oestrus in cattle, dose control is exerted over the time at which a unit dose is delivered and over the duration and over the dose volume of a continuous series of pulsatile doses for delivery of the progesterone formulation and single release doses of the oestrogen and prostaglandin formulations.

### EXAMPLE 8

### A Ten Day Treatment Period including an Eight Day Progesterone Delivery Programme

This discussion focuses on the preferred delivery regime for the progesterone active in an eight day delivery programme and variations to the design of the delivery device (both the delivery apparatus/reservoirs and the control means) to accommodate these changes.

As part of a preferred eight day progesterone delivery programme the need was identified to increase the size of the bellows of the delivery device containing the progesterone formulation and to dose the progesterone solution more frequently as a result of the short half-life of the hormone.

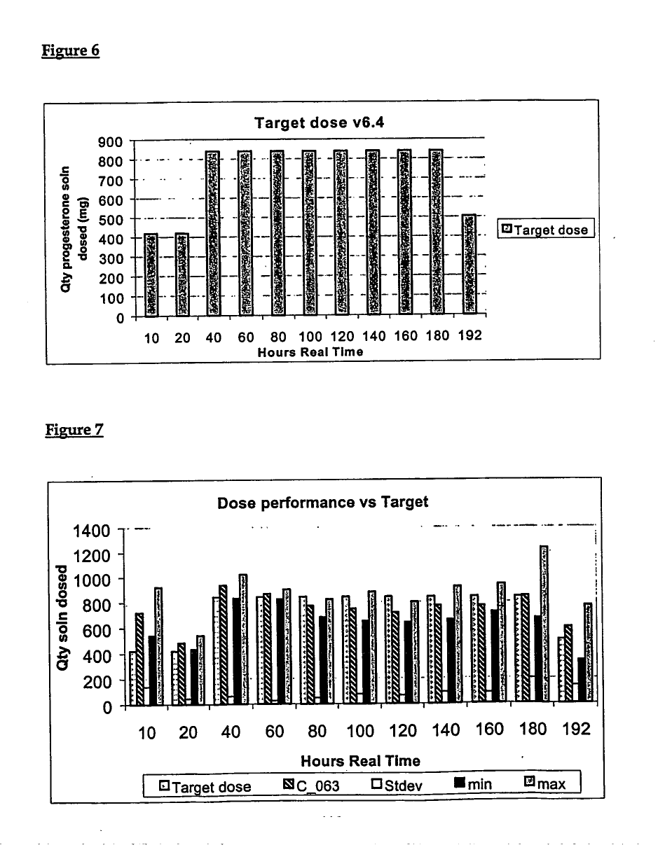

One embodiment of the delivery device was calibrated to the following specification for pulsatile dosing of progesterone as is shown in FIGS. 7 to 9. The software program used in this example is the version relevant to the programmes of events for progesterone dosing illustrated previously in Tables 2 and 3.

Through the development of improved formulations and extensive in vivo assessment a modified drug regime was developed. The delivery timing of oestrogen as well as progesterone was changed. It was also observed that dosing prostaglandin on Day 6 was possibly contributing to a premature drop in progesterone serum values in specific cows. Accordingly, dosing the prostaglandin approaching Day 8, but prior to the cessation of progesterone delivery, was implemented. Consequently the delivery regime for one embodiment utilising an 8-day progesterone delivery programme is shown in FIG. 10.

Progesterone Solutions

As a consequence of the decision to remove benzyl alcohol from the delivery device, at least two other proprietary solutions for dispensing progesterone were developed. These have been previously discussed in Example 4(B), under the heading of alternative carrier solutions and the results of various trials have been presented in FIGS. 3 to 5.

This discussion however relates to an 8-day progesterone priming programme in combination with other preferred actives, such as the oestrogen formulations (for example, oestrogen 17β) and the prostaglandin formulation.

The intravaginal device used with this embodiment of the invention relies on the use of modified dosing software and outlet configurations to effect the required ten day treatment period with an eight day progesterone programme for delivery of the various hormone actives into the animal at the preferred time, in preferred concentrations, and in preferred amounts.

The 8-day progesterone programme resulted from trials with ovarectomised cows in which it was identified that during a 10-day progesterone programme there was a ½ life issue, with the progesterone being metabolised by the cow quicker than the preferred delivery device was dosing. In the 10-day programme progesterone is dosed, on average, hourly for the first 7 days of the treatment programme before reducing to dose intervals of 28 minutes and less for the remaining 3 days of the programme.

Consequently, in some embodiments and with some animals serum progesterone levels may not be increased sufficiently and/or may not be maintained at a level sufficient for foflicular turnover and development to occur in entire cows. In the 10-day programme blood serum progesterone levels in the cow rapidly increased (within 1 hour of dosing commencing) to preferably no less than 2 ng/1 nl (for deeply anoestrus animals, 2 ng/ml or less of progesterone may not be sufficient, or some animals may metabolise exogenous progesterone faster than others) and remained close to that level for the initial 7 days. The level of progesterone was maintained, via the supply of exogenous progesterone, at a minimum threshold even for animals producing their own endogenous progesterone.

It is preferable to effect elevated blood serum levels of progesterone within 20 minutes of the delivery device being switched on. Initial elevated levels of progesterone are required to effect control on the current fertility status of all animals being treated. The delivery regime effectively loads up the progesterone receptors and causes the hypothalamus to recognise the high progesterone levels. All animals therefore preferably have the same progesterone level. This is the first step in resetting the follicular waves.

When the dose interval of progesterone drops to 28 minutes and less, blood serum progesterone levels have been recorded as dramatically increasing to around 8 ng/ml. They tend to remain at that level until cessation of progesterone pumping where the progesterone levels rapidly decreases to basal levels within 24 hours. This profile has been observed for all cows on a 10-day treatment as previously described, irrespective of the quantities of progesterone delivered (1.8 grms of p4±0.2).

The levels of progesterone are preferably maintained at the elevated level for a period of time following the first release of oestrogen into the animal. This is because if the progesterone levels are too low those animals approaching oestrus will go in to oestrus. The high progesterone levels prevents this from occurring. Effecting elevated progesterone levels is essentially the first step in resetting the animal's follicular waves.

Further, if the preferred oestrogen absorption is not achieved initiation of the LH surge necessary for the onset of oestrus and subsequent ovulation will not occur. The oestrogen used with the 10-day progesterone delivery programme previously described in Example 5 is in the form of a benzoate. For the 8-day progesterone delivery programme oestradiol 17β was used and as such is the active form the oestradiol benzoate is metabolised into and that is typically measured in the blood serum assay.

It is preferable that a spike of oestrogen be effected to ensure all animals revert to a common starting point. The oestrogen spike effects release of mature eggs and conditions the ovary to a state where it is ready to produce a new egg.

In the present example of controlling oestrus as a biological function, it should be appreciated that non-cycling animals do not display follicular waves. However, it is difficult to determine the exact breeding status of each animal. Some may be sporadically anoestrus, some fully anoestrus, and some may have silent heats.

In the present application, all animals are treated irrespective of their possible breeding status to ensure all animals are at a common starting point. Anoestrus cows do not have follicular waves, but the progesterone treatment effects these again. For cycling cows, the progesterone treatment resets the follicular waves.

On the basis of prior art assessments that 12 pg/ml of oestrogen was necessary, in spike form, to promote oestrus behaviour, the use of oestradiol benzoate was considered not to be the most effective oestrogen, due to its hydrophobic nature and slow release characteristics. Accordingly, the alternate 17β oestrogens were tested for use with this invention as another preferred form of oestrogen. Certain penetrants and excipients were also assessed and are discussed (where relevant) in relation to their assistance in achieving the desired serum profile.

One preferred dose of the oestradiol 17β active (as identified specifically in this specification) used for the current application is 2 mg. Nevertheless, as can be appreciated, different treatments may require different preferred doses of oestradiol. For example, the dose may fall within the range ≧0.5 mg to ≧7 mg of oestradiol with a cyclodextrin carrier.

When oestradiol 17-β of the present invention is coupled with the preferred cyclodextrin carrier (which for the purpose of this discussion is hydroxpropyl 17β-cyclodextrin) a very rapid absorption is observed, with the corresponding peak in blood serum levels to achieve the desired results. Efficacious results have been obtained whether the oestradiol 17-β formulation is in solid (a tablet) or fluid form.

Serum oestradiol, from basal levels, reached peak values within 2 to 3 hours, with consistently well defined spikes. A 1 mg dose produced maximum mean values of 130-180 pg/ml at 100-130 minutes after treatment, while a 2 mg dose led to maxima of 180 to >250 pg at 120-150 minutes, as illustrated in FIG. 11.

The values attained exceeded the peak plasma concentration maximums recorded in the prior art of 8 to 13 pg/ml obtained approximately 2 hours following administration of 1 mg oestradiol benzoate administered by intramuscular injection, or peak plasma concentrations of between 10-20 pg/ml within four hours following administration of 7.2 mg of oestradiol 17β administered intravaginally.

A primary objective is achieving a spike in plasma oestradiol to be an effective oestradiol surge for the purpose of stimulating (behavioural and/or functional) oestrus response. That the levels remain elevated for at least 24 hours, is not an important factor. Rather, a pronounced oestradiol spike (for short duration), total bioavailability, or period above a critical value, may be better correlated with clinical efficacy for either follicular atresia or stimulation of oestrus.

In further embodiments of the present invention two separate doses of the oestrogen (such as oestradiol 17-β) formulation may be administered to effect the desired outcome. The first is preferably administered within approximately 2 hours following device activation, whilst the second is delivered on or about day nine (9) of a 10 day treatment programme.

It has been noted that increasing the amount of oestradiol 17β, and/or increasing the amount of cyclodextrin within the preferred weight to weight ratio range of oestradiol:cyclodextrin does have a significant affect on the time to maximum plasma concentrations of the oestradiol. In addition there is a notable affect on the actual maximum plasma concentration of the oestradiol.

At approximately day 7-8 of the eight day progesterone dosing programme (where the total treatment period is 10 days) the prostaglandin/carrier formulation is administered from the device. The introduction of prostaglandin removes the corpus lutea from the animals and prevents the animals from producing any endogenous progesterone. By day 8 the delivery of progesterone is ceased and the animal no longer produces her own endogenous progesterone due to the prostaglandin formulation dosed.

Trials have shown that in animals treated with ≧1 ng/ml of prostaglandin, ovulation still occurs, but they demonstrate suppressed overt oestrus.

Ovulation in such animals is dependent on the second spike of oestrogen delivered via the delivery regime which stimulates the lutenising hormone (LH) surge and release of follicle stimulating hormone (FSH)—basically forcing ovulation.

In animals treated with ≦1 ng/ml they are truly in oestrus and have a very high expression of overt oestrus.

Objectives

In establishing an 8-day progesterone treatment programme the objectives were:
a) delivery of the formulations using the preferred delivery device; and
b) using the standard prostaglandin treatment, in combination with different forms of oestrogen,
c) effective control and synchronisation of oestrus in empty dairy cows ten days after commencement of treatment.

Twenty-one non-pregnant and physiologically normal, mixed age, adult “carry over” dairy cows were used to test the invention. These cows were regarded as being “hard to breed” as they had failed to conceive during normal mating programs during the previous 6 months.

Assessment of ovarian activity at the beginning of the trial was accomplished by transrectal B-mode ultrasound (Aloka 210 with 5 MHz probe) in all cows to identify ovarian structures. Ovaries were classed by the presence and location of small (<5 mm), medium (5-10 mm) or large (>10 mm) follicles with the presence or absence of a corpus luteum identified. All assessments were equally balanced across 3 treatment groups so each ovarian condition was exposed to all treatments, as follows:

Treatment 1 Contained

2 grams of progesterone in solution
1 dose of 240 μg of prostaglandin,as sodium cloprostenol with carrier/excipient in tablet form
1 dose of 7 mg estradiol 17β in tablet form with carrier/excipient
1 dose of 2 mg estradiol 17β in tablet form with carrierlexcipient
   Treatment 2 Contained
2 grams of progesterone in solution
1 dose of 240 μg of prostaglandin as sodium cloprostenol with carrier/excipient in tablet form
1 dose of 7 mg estradiol 17β in capsule form+carrier
1 dose of 2 mg estradiol 17β in capsule form+carrier.

Capsule size, in order to be administered automatically on day 0 and day 9, need to fit into the unit dose chambers in the delivery device used. For trial purposes they were delivered manually at the same time as they would have been if delivered automatically by the delivery device.

Treatment 3 Contained

2 grams of progesterone in solution.
1 dose of 240μg prostaglandin as sodium cloprostenol with excipients in tablet form.

This group of cows were treated with oestradiol benzoate (5 mg/ml, Intervet, Australia) with 1.2 mls injected intramuscularly on day 0 and 0.4 mls on days 9.

Transrectal B-mode ultrasound was used to identify the likely ovulatory dominant follicle on day 10 with confirmation of ovulation 2 days later.

Summary of Results

Day 0: Ovarian Scan and Delivery Device Insert

Fifteen cows were assessed as havsng small (<5 mm) or medium (5-10 mm) follicles present, 6 had large (>10 mm) follicles. Of the total group 11 cows were also identified with the presence of a corpus luteum (9 were classed as large CL's (15 mm), 2 small (<10 mm)). See Table 7 below.

TABLE 7
Treatment allocation based on ovarian condition day 0

| | Ovary Status | | |
|---|---|---|---|
| Tag No | Left | Right | Treatment |
| 17 | SF | LF | 2 |
| 28 | LF | CL | 3 |
| 37 | SCLMF | SF | 1 |
| 83 | CLLF | SF | 2 |
| 85 | MF | MF | 1 |
| 109 | MF | CL | 1 |
| 125 | MF | SF | 3 |
| 181 | SF | MF | 3 |
| 233 | SF | HCL | 3 |
| 243 | LF | CL | 2 |
| 247 | SF | CL | 1 |
| 280 | SF | LF | 3 |
| 307 | SF | CL | 3 |
| 338 | LF | SFSCL | 1 |
| 425 | SF | SF | 2 |
| 431 | MF | MF | 3 |
| 448 | SF | MF | 2 |
| 458 | SF | MF | 1 |
| 485 | CLMF | SF | 2 |
| 472 | CL | MF | 1 |
| 473 | MF | MF | 2 |

CL = Corpus Luteum;
SF = Small follicle;
MF = Medium;
LF = Large

Delivery Device Retention and Adverse Physiological Effect

The delivery devices were removed from 20 cows on day 10 (96% retention), with only one loss of the delivery device recorded. Each cow was examined and a score attributed for irritation, mucous development and damage to the dorsal vulva commisure. There was some minor evidence, in only 4 cows, of vaginal irritation or damage to the vulva commisure at the time of removal, but this was not considered to be of concern.

Irritation of the Vaginal Mucosa

TABLE 8
Irritation score

| Irritation | Mucous |
|---|---|
| # Cows | # Cows |
| 4 | 4 |
| Range 1-2 | Score 1 |
| 0 = None | 5 = Severe |

Four cows identified with minor signs of irritation to the vulva or vagina in Table 8. Three of them had a score of 2, one a score of 1. All others had no visible signs of irritation. The presence of mucous was identified in only 4 cows each with a score Of 1, where 0 equated to no mucous.

Synchrony of Oestrous Day 10

Oestrous activity was monitored throughout the trial and on day 10. Oestrus was synchronised in 86% (n=18) of the total group (n=21) on day 10. The results are shown in FIG. 14.
72% of treatment 1 showed overt oestrus.
100% of treatment 2 showed overt oestrus.
86% of treatment 3—showed overt oestrus.
   Ovarian Scans on Day 10

B-mode ultrasound (Aloka 210 with a 5MHz probe) was used in all cows to identify ovarian structures on day 10 (day of delivery device removal), to confirm the presence and location of a dominant, large, ovulatory follicle, plus identification of any other ovarian structures that may be present. The dominant follicle was defined as the largest follicle present on either ovary.

Of the 20 cows examined on day 10, 90% of the trial herd had a large, >15 mm diameter dominant follicle present and the majority of these cows exhibited overt oestrus activity. All cows were re-examined using ultrasound, 2 days later to confirm ovulation. Results are shown in Table 9.

TABLE 9

| | Large F > 15 mm |
|---|---|
| Treatment 1 | 5 Cows |
| Treatment 2 | 7 Cows |
| Treatment 3 | 6 Cows |

Ovulation Day 12

Table 10 shows rescanning results of cows on day 12 to confirm ovulation and identify presence of a corpus haemorrhagum, (CH). Ovulation was confirmed by disappearance of previously identified large follicle. In two thirds of the cows a corpus haemorrhagum was identified.

TABLE 10
Summary of ovarian activity

| | Standing | Large F | Ovulation | CH |
|---|---|---|---|---|
| | heat d10 | d10 | d12 | d12 |
| Trt 1 | 5 | 5 | 4 | 4 |
| Trt 2 | 7 | 7 | 7 | 6 |
| Trt 3 | 5(+1 silent heat) | 6 | 5 | 4 |

Delivery Device Performance

All delivery devices functioned as expected with all of the pots firing with the oestrogen and prostaglandin tablets being expelled. Each delivery device was loaded with 2.1 grams of progesterone in solution. The average amount of progesterone dosed was 1.84 grams (1.71 grams-1.9 grams). The maximum amount of progesterone that could be dosed was about 1.9 grams. The average residual, undosed, progesterone in the reservoir was 0.34 gms (0.29-0.45 gms).

This trial demonstrated the integrity of the delivery device with the precise delivery of the respective hormones and pulsatile dosing of the progesterone being able to synchronise oestrus in 85% of the herd. The identification of an ovulation and subsequent appearance of luteal tissue confirmed this. The intravaginal delivery of oestrogen via treatment 1 and 2 produced comparable results to the delivery of oestrogen via intra-muscular delivery. An 8-day progesterone delivery program in combination with the prostaglandin and oestradiol treatments synchronised oestrus and ovulation in a group of difficult to breed cows.

### EXAMPLE 8

This product incorporates four formulations administered in fixed ratios as part of a single treatment, in one preferred embodiment of the invention. These are described below.

Formulation 1

Two grams progesterone are provided in 7 millilitres of solution

| Ingredient name | Function |
|---|---|
| Progesterone | Ovarian suppressant |
| Propylene glycol | Cosolvent |
| Phenylethanol | Solvent |

Formulation 2

Seven milligrams oestradiol are provided in one 100 milligram tablet

| Ingredient name | Function |
|---|---|
| Beta-oestradiol | Follicular atresic |
| β cyclodextrin | Complexing agent |
| Cellulose | Binder |
| Colours | Colourant |
| Colloidal silica | Flow agent |
| Magnesium stearate | Lubricant |

Formulation 3

Two milligrams of oestradiol are provided in one 60 mg tablet

| Ingredient name | Function |
|---|---|
| Beta-oestradiol | Oestrogenic |
| β cyclodextrin | Complexing agent |
| Cellulose | Binder |
| Colour | Colourant |
| Colloidal silica | Flowing agent |
| Magnesium stearate | Lubricant |

Formulation 4

240 micrograms of cloprostenol are provided in one 60 mg tablet

| Ingredient name | Function |
|---|---|
| Cloprostenol sodium | Luteolytic |
| Cellulose | Binder |
| Colloidal silica | Flowing agent |
| Magnesium stearate | Lubricant |

### EXAMPLE 9

### Quality Control of Drug Formulations

Before filling the delivery device with the formulations (active/carrier) the actives undergo quality control tests.

Each batch of the progesterone active is and, in application will continue to be, filtered through a 10μ filter to remove all impurities likely to create blockages in the pumping mechanism of the device. During use of the formulations regular, monthly testing is proposed specifically for progesterone for stability and correct concentration.

All hormonal solutions have been put through microbiological challenge tests to ensure the formulations could withstand bacterial growth over the rested shelf life time span.

A sample was taken from each batch of progesterone and cultured for Microbiological purity, testing for salmonella and coliforms (plate count technique).

The samples were analysed using BP 1988 Volume II Methodology Appendix XVIC. Cultures were maintained in accordance with the recommendations of the curator of the culture collections centre. The results of the tests are presented in Table 17.

The following organisms were used in the tests:
1. Aspergillus niger (ATCC 16404)
2. Candida albicans (ATCC 10231)
3. Pseudomonas aeruginosa (ATCC 15442)
4. Staphylococcus aureus (ATCC 3022)

The number of bacterial organisms recovered per ml was reduced by a factor of not less than 10² within 7 days of challenge and there was no increase thereafter. This test was completed at the beginning of the prototypic phase of the product development and as all tests showed a negative result there has not been a requirement to repeat the tests. Samples were also tested for their ability to maintain yeast or mould preparations. No increase was detected per millilitre in the number of yeast/mould organisms within 14 days of challenge or thereafter.

A separate challenge analysis was carried out on the cloprostenol solution (prostaglandin) and tested for bactericidal properties against faecal coliforms. The solution was challenged with a stock culture of Escherichia coli (E. Coli).

The cloprostenol solution was innoculated on two separate occasions with high numbers of E. coli at approximately 10⁶ per gram of sample. Even just after innoculation, the presence of E. coli was almost undetectable. After seven and 15 days, the E. coli were less than 10 per gram, confirming the bactericide properties of the cloprostenol solution against faecal coliforms.

TABLE 17
Results of Microbiological Purity/tests for Formulations

| | | | | |
|---|---|---|---|---|
| Zero hour | 48 hours | 7 days | 14 days | 28 days |

Progesterone Solution

| | | | | | |
|---|---|---|---|---|---|
| Aspergillus niger | 1.3 × 10 | <1.0 × 10 | <1.0 × 10 | <1.0 × 10 | <1.0 × 10 |
| Control | 5.0 × 10 | 1.0 × 10 | 7.0 × 10 | 2.6 × 10 | 5.1 × 10 |
| Candida albicans | <1.0 × 10 | <1.0 × 10 | <1.0 × 10 | <1.0 × 10 | <1.0 × 10 |
| Control | 2.5 × 10 | 4.6 × 10 | 8.3 × 10 | 4.1 × 10 | 1.7 × 10 |
| Staphylococcus aures | <1.0 × 10 | <1.0 × 10 | <1.0 × 10 | <1.0 × 10 | <1.0 × 10 |
| Control | 1.3 × 10 | 1.6 × 10 | 2.6 × 10 | 1.0 × 10 | 5.3 × 10 |
| Pseudomonas aeruginosa | <1.0 × 10 | <1.0 × 10 | <1.0 × 10 | <1.0 × 10 | <1.0 × 10 |
| Control | 1.8 × 10 | 3.4 × 10 | 6.0 × 10 | 4.0 × 10 | 1.9 × 10 |

Oestradiol Benzoate

| | | | | | |
|---|---|---|---|---|---|
| Aspergillus niger | <1.0 × 10 | <1.0 × 10 | <1.0 × 10 | <1.0 × 10 | <1.0 × 10 |
| Control | 5.0 × 10 | 1.5 × 10 | 7.0 × 10 | 2.6 × 10 | 5.1 × 10 |
| Candida albicans | <1.0 × 10 | <1.0 × 10 | <1.0 × 10 | <1.0 × 10 | <1.0 × 10 |
| Control | 2.5 × 10 | 4.6 × 10 | 8.3 × 10 | 4.1 × 10 | 1.7 × 10 |
| Staphylococcus aures | <1.0 × 10 | <1.0 × 10 | <1.0 × 10 | <1.0 × 10 | <1.0 × 10 |
| Control | 1.3 × 10 | 1.6 × 10 | 2.6 × 10 | 1.0 × 10 | 5.3 × 10 |
| Pseudomonas aeruginosa | <1.0 × 10 | <1.0 × 10 | <1.0 × 10 | <1.0 × 10 | <1.0 × 10 |
| Control | 1.8 × 10 | 3.4 × 10 | 6.0 × 10 | 4.0 × 10 | 1.9 × 10 |

Oestradiol Benzoate 2

| | | | | | |
|---|---|---|---|---|---|
| Aspergillus niger | <1.0 × 10 | <1.0 × 10 | <1.0 × 10 | <1.0 × 10 | <1.0 × 10 |
| Control | 5.0 × 10 | 1.0 × 10 | 7.0 × 10 | 2.6 × 10 | 5.1 × 10 |
| Candida albicans | <1.0 × 10 | <1.0 × 10 | <1.0 × 10 | <1.0 × 10 | <1.0 × 10 |
| Control | 2.5 × 10 | 4.6 × 10 | 8.3 × 10 | 4.1 × 10 | 1.7 × 10 |
| Staphylococcus aures | <1.0 × 10 | <1.0 × 10 | <1.0 × 10 | <1.0 × 10 | <1.0 × 10 |
| Control | 1.3 × 10 | 1.6 × 10 | 2.6 × 10 | 1.0 × 10 | 5.3 × 10 |
| Pseudomonas monas | <1.0 × 10 | <1.0 × 10 | <1.0 × 10 | <1.0 × 10 | <1.0 × 10 |
| Control | 1.8 × 10 | 3.4 × 10 | 6.0 × 10 | 4.0 × 10 | 1.9 × 10 |

Cloprostenol Sodium (PG)

| | | | | | |
|---|---|---|---|---|---|
| Aspergillus niger | 4.6 × 10 | <1.0 × 10 | <1.0 × 10 | <1.0 × 10 | <1.0 × 10 |
| Control | 5.0 × 10 | 1.0 × 10 | 7.0 × 10 | 2.6 × 10 | 5.1 × 10 |
| Candida albicans | 5.7 × 10 | <1.0 × 10 | <1.0 × 10 | <1.0 × 10 | <1.0 × 10 |
| Control | 2.5 × 10 | 4.6 × 10 | 8.3 × 10 | 4.1 × 10 | 1.7 × 10 |
| Staphylococcus aures | <1.0 × 10 | <1.0 × 10 | <1.0 × 10 | <1.0 × 10 | <1.0 × 10 |
| Control | 1.3 × 10 | 1.6 × 10 | 2.6 × 10 | 1.0 × 10 | 5.3 × 10 |
| Pseudomonas monas | <1.0 × 10 | <1.0 × 10 | <1.0 × 10 | <1.0 × 10 | <1.0 × 10 |
| Control | 1.8 × 10 | 3.4 × 10 | 6.0 × 10 | 4.0 × 10 | 1.9 × 10 |

### EXAMPLE 10

This example is a data sheet for one version of the preferred intravaginal breeding device, employing preferred formulations, to synchronising oestrus in cows with reference to a 12 day programme. However, other programmes are available and the information provided below will vary accordingly.

1. Trade Name of Product: (The name of the preferred intravaginal breeding device).

2. Description: Electronically timed drug release unit that dispenses different hormones under a predetermined sequence. Hormones used are forms of natural progesterone, oestradiol benzoate and prostaglandin F₂α (cloprostenol).

3. Mode of Action: Using 3 (three) hormones in combination to synchronise follicle waves, with the objective of having a newly formed mature dominant follicle ovulate prior to the time of planned insemination.

4. Indications: To synchronise oestrus in cycling and non-cycling dairy or beef cows for fixed time planned insemination.

5. Contraindications: Do not use in pregnant animals as abortion may result.
Not suitable for animals with reproductive disorders or abnormal vaginas. If animals are under nutritional stress or suffering from illness or debilitation the results of the treatment may be poor.
Do not use on cows within 30 days post calving without prior veterinary advice and examination.

6. Dosage and Administration:
One device inserted vaginally for 12 days, 12 days prior to insemination. Used in cycling or anoestrus, lactating or non lactating dairy or beef cows dispenses the following doses of hormone:
Day 1: insert preferred intravaginal breeding device, approximately 42 mg±5% Progesterone solution dosed 2 hourly+a spike release of 6.8 mg Oestradiol Benzoate.
Day 2-10: 42 mg±5% Progesterone solution dosed 2 hourly.
Day 6: Spike release of 240 mcg Prostaglandin (Cloprostenol Sodium).
Day 11: Spike release of 0.9 mg Oestradiol Benzoate.
Day 12: Remove preferred intravaginal breeding device and inseminate all cows in treatment group.

7. Warnings to Handler/Operator:
Once the preferred intravaginal breeding device is switched on, the first pot is released within 20 minutes, and progesterone commences dosing within 2 hours.

A similar data sheet for use with other programmes using a variation of the intravaginal breeding device and controlled delivery of the hormone formulations for synchronising oestrus in dairy or beef heifers for fixed time planned insemination would be available with the following differences specific to identify the applicable programme. For example:
1. Trade Name of Product: alternative preferred intravaginal breeding device
2. Indications: To synchronise oestrus in dairy or beef heifers for fixed time planned insemination.
3-7: These may be similar to the above data, where appropriate, or the information would be varied as applicable to the particular device and/or the delivery regime.

The important features of the invention are the autonomous delivery of a range of tailored formulations at one site, in accordance with a programmed delivery regime, from a delivery device capable of housing the formulations separately and effecting delivery of the formulations. The formulations are delivered via the delivery apparatus and the control means of the delivery device at the precise time, in the preferred quantities and/or concentrations, for the preferred duration, in the preferred sequence, to effect control of a biological function.

Aspects of the present invention have been described by way of example only and it should be appreciated that modifications and additions may be made thereto without departing from the scope thereof, as defined in the appended claims.

1. A series of formulations, each including at least one active component as herein defined, in combination with at least one facilitating transfer agent as herein defined, and optionally an excipient, the formulations compatible for delivery at substantially the same site during an administration regime, and which work in conjunction to achieve a particular physiological change associated with a biological function.

2. A series of formulations as claimed in claim 1 in which the biological function comprises a reproductive process.

3. A series of formulations as claimed in claim 2 in which the reproductive process is the synchronisation of oestrus.

4. A series of formulations as claimed in claim 2 in which the active is selected from a group including progesterone, an oestrogen, a prostaglandin.

5. A series of formulations as claimed in claim 4 in which the active is selected from at least one of a derivative, an analogue thereof of any one of said group.

6. A series of formulations as claimed in claim 1 wherein said at least one facilitating transfer agent is any means in solid or fluid form (such as powder, tablet, liquid, paste, gas, and so forth) to assist the transfer, transport, absorption, solubility of an active across a physiological barrier (such as a membrane), to effect improved bioavailability of the active to the animal (such as increased levels in the blood).

7. A series of formulations as claimed in claim 6 in which said at least one facilitating transfer agent used in combination with said at least one of said formulations is a cyclodextrin, a suitable cyclodextrin derivative displaying the preferred properties, or a substitute compound displaying the preferred properties.

8. A series of formulations as claimed in claim 1 in which said formulations are suitable for delivery by a device of the type including a body, the body capable of housing delivery apparatus capable of actively being controlled to autonomously deliver at least one substance into a cavity, said delivery apparatus including dedicated pressure systems to deliver the formulations from independent reservoirs via associated outlet(s), said formulations ranging in form from substantially fluid to substantially solid, the device also including programmable control means capable of initiating and regulating delivery of the formulations in accordance with a preferred delivery regime, the body further including retention apparatus capable of effecting retention of the device within the cavity.

9. A series of formulations as claimed in claim 5 in which an active component is selected from the group: progesterone, oestradiol benzoate (oestra-1,3,5 (10)-triene-3,17β-diol 3-benzoate), oestradiol hemihydrate (oestra-1,3, 5 (10)-triene-3, 17β-diol or oestradiol 17β), and cloprostenol sodium (an analogue of Dinoprost prostaglandin F₂α).

10. A series of formulations as claimed in claim 9 in which there are three formulations, each formulation including one of: progesterone, oestradiol benzoate (oestra-1,3, 5 (10)-triene-3,β-diol 3-benzoate) and cloprostenol sodium.

11. A series of formulations as claimed in claim 9 in which there are three formulations, each formulation including one of: progesterone, oestradiol hemihydrate (oestra-1,3,5 (10)-triene-3,17β-diol or oestradiol 17β) and cloprostenol sodium.

12. A series of formulations as claimed in claim 2 in which one formulation includes an oestrogen as an active component in combination with a facilitating transfer agent comprising at least one of: a cyclodextrin, a suitable cyclodextrin derivative displaying the preferred properties, or a substitute compound displaying the preferred properties.

13. A series of formulations as claimed in claim 2 in which said one formulation includes as a facilitating transfer agent any one of: at least hydroxypropyl-beta-cyclodextrin (HPβCD), dimethyl-beta-cyclodextrin and hydroxyethyl-beta-cyclodextrin being water soluble and forming very soluble inclusion complexes.

14. A series of formulations as claimed in claim 1 when used in conjunction to effect said biological function.

15. A series of formulations as claimed in claim 3 when used in conjunction to effect synchronisation of oestrus.

16. A series of formulations as claimed in claim 1 when used to implement the method of affecting a biological function consisting of automated release of active components, the method including an administration regime consisting of at least a first delivery phase for the release of at least a first active component, as well as a second delivery phase for release of at least a second active component, each phase consisting of parameters including one or more of release time, duration, magnitude; the release quantity versus time profiles on said two delivery phases differing, and wherein said first and second active components co-operate to achieved a desired outcome.

17. A series of formulations as claimed in claim 1 wherein the excipients optionally include at least one of preservatives, free-flowing agents, binders, colourants, penetration aids/carriers.

18. A series of formulations as claimed in claim 3 in which to effect synchronization of oestrus the penetration aids/carriers improves the transfer of the active through the vaginal mucosa.

19. A series of formulations as claimed in claim 4 wherein the penetration aid/carrier for effecting improved transfer of the progesterone through the vaginal mucosa includes at least one of magnesium stearate, a fatty acid.

20. A formulation for in situ release in an animal including at least one active component as herein defined for affecting a biological function associated with reproductive processes, in combination with at least one facilitating transfer agent as herein defined, and optionally one or more excipients; said facilitating transfer agent including a cyclodextrin, including a suitable cyclodextrin derivative displaying the preferred properties, a solvent.

21. A formulation for in situ release in an animal as claimed in claim 20 when used according to the method of affecting a biological function consisting of automated release of active components, the method including an administration regime consisting of at least a first delivery phase for the release of at least a first active component, as well as a second delivery phase for release of at least a second active component, each phase consisting of parameters including one or more of release time, duration, magnitude; the release quantity versus time profiles on said two delivery phases differing, and wherein said first and second active components co-operate to achieved a desired outcome.

22. A formulation for in situ release in an animal including at least one active component as herein defined for affecting a biological function, in combination with at least one facilitating transfer agent as herein defined, and optionally one or more excipients; said formulation compatible with at least a second formulation consisting of at least one active component as herein defined for affecting a biological function or any stage of a process thereof, in combination with at least one facilitating transfer agent as herein defined, and optionally one or more excipients, for co-administration or co-delivery within the same administration regime duration at the same site; the two formulations being directed to achieve the desired outcome.

23. A formulation for in situ release in an animal as claimed in claim 22 in which a biological function is a reproductive process.

24. A formulation for in situ release in an animal as claimed in claim 23 in which the reproductive process is the synchronisation of oestrus.

25. A formulation for in situ release in an animal as claimed in claim 22 when used according to the method of affecting a biological function consisting of automated release of active components, the method including an administration regime consisting of at least a first delivery phase for the release of at least a first active component, as well as a second delivery phase for release of at least a second active component, each phase consisting of parameters including one or more of release time, duration, magnitude; the release quantity versus time profiles on said two delivery phases differing, and wherein said first and second active components co-operate to achieved a desired outcome.

26. A formulation for in situ release in an animal as claimed in claim 20 which includes as an active component a prostaglandin or derivative thereof, and a cyclodextrin, including a suitable cyclodextrin derivative displaying the preferred properties, as a facilitating transfer agent.

27. A formulation for in situ release in an animal as claimed in claim 20 which includes as an active component a progesterone or derivative thereof, and a cyclodextrin, including a suitable cyclodextrin derivative displaying the preferred properties, as a facilitating transfer agent.

28. A formulation for in situ release in an animal as claimed in claim 20 which includes as an active component an oestrogen or derivative thereof, and a cyclodextrin, including a suitable cyclodextrin derivative displaying the preferred properties, as a facilitating transfer agent.

29. A formulation for in situ release in an animal as claimed in claim 26 in which the prostaglandin is sodium cloprostenol.

30. A formulation for in situ release in an animal as claimed in claim 26 in which the cyclodextrin is cyclodextrin HPβCD.

31. A formulation for in situ release in an animal as claimed in claim 26 in which the prostaglandin or derivative thereof and the cyclodextrin occurs in a weight to weight ratio of between 1:7 to 1:18 active:facilitating transfer agent.

32. A formulation for in situ release in an animal as claimed in claim 26 in which the prostaglandin or derivative thereof and the cyclodextrin occurs in a weight to weight ratio of between 1:11 to 1:14 active:facilitating transfer agent.

33. A formulation for in situ release in an animal as claimed in claim 31 in which the prostaglandin when combined with cyclodextrin HPβCD is available for use in a delivery regime for synchronising oestrus in an animal in a dose range using between 500 μg and 1.5 mg sodium cloprostenol per unit dose.

34. A formulation for in situ release in an animal as claimed in claim 33 in which the prostaglandin unit dose for heifers is 240-250 μG sodium cloprostenol plus HPβCD and for larger cattle species such as buffalo a unit dose of ≧2.5 mg is optionally used to effect optimal results.

35. A formulation for in situ release in an animal as claimed in claim 34 in which the prostaglandin and the cyclodextrin formulation on delivery effects a prostaglandin blood serum level directed to synchronising oestrus in cows.

36. A formulation for in situ release in an animal as claimed in claim 35 in which the prostaglandin and the cyclodextrin formulation is required to be administered as part of a delivery regime to synchronise oestrus in cows at a point in advance of cessation of any exogenous progesterone administration to ensure endogenous progesterone will not mask the precipitous fall in serum progesterone resulting from this cessation.

37. A formulation for in situ release in an animal as claimed in claim 36 in which the prostaglandin and the cyclodextrin formulation is required to be administered in accordance with the delivery regime on approximately day 7.5 of a delivery regime.

38. A formulation for in situ release in an animal as claimed in claim 27 in which the progesterone or derivative thereof and the cyclodextrin occurs in a weight to weight ratio of between 1:8 to 1:17 active:facilitating transfer agent.

39. A formulation for in situ release in an animal as claimed in claim 27 in which the progesterone or derivative thereof and the cyclodextrin occurs in a weight to weight ratio of between 1:11 to 1:14 active:facilitating transfer agent.

40. A formulation for in situ release in an animal as claimed in claim 27 in which the cyclodextrin is cyclodextrin HPβCD.

41. A formulation for in situ release in an animal as claimed in claim 38 in which the progesterone when combined with cyclodextrin HPβCD is available for use in a delivery regime for synchronising oestrus in an animal in a dose range using between 0.5 gm to 2.2 gm of progesterone.

42. A formulation for in situ release in an animal as claimed in claim 20 which includes as an active component a progesterone or derivative thereof, and a solvent as a facilitating transfer agent.

43. A formulation for in situ release in an animal as claimed in claim 42 in which the solvent facilitating transfer agent is any one of benzyl alcohol, marlophenol, propylene glycol and phenylethanol, ethanol, a glycol, water.

44. A formulation for in situ release in an animal as claimed in claim 42 in which the progesterone or derivative thereof and benzyl alcohol occurs in a weight to volume ratio of between 38-40% active:facilitating transfer agent.

45. A formulation for in situ release in an animal as claimed in claim 44 in which the progesterone when combined with benzyl alcohol is available for use in a delivery regime for synchronising oestrus in an animal at a maximum dose rate per 24 hours during the treatment period of approximately 200 mg/day via intravaginal administration.

46. A formulation for in situ release in an animal as claimed in claim 42 in which the progesterone or derivative thereof and any one of marlophenol, propylene glycol and phenylethanol, ethanol and water occurs in a weight to volume ratio of between 70%-99.8% active:facilitating transfer agent.

47. A formulation for in situ release in an animal as claimed in claim 46 in which the progesterone when combined with a solvent is available for use in a delivery regime for synchronising oestrus in an animal at a dose rate of up to 42 mg of progesterone solution every 2 hours.

48. A formulation for in situ release in an animal as claimed in claim 42 in the facilitating transfer agent is optionally used with a penetration aid to further effect improved transfer of the progesterone through the vaginal mucosa.

49. A formulation for in situ release in an animal as claimed in claim 48 in which the penetration aid replaces a percentage volume of the solvent.

50. A formulation for in situ release in an animal as claimed in claim 49 wherein when the penetration aid is a fatty acid, approximately 33% of the volume of the solvent is replaced with the fatty acid.

51. A formulation for in situ release in an animal as claimed in claim 38 in which the progesterone and the cyclodextrin formulation on delivery effects a progesterone blood serum level directed to controlling oestrus in cattle of 2-8 ng/ml within 100 minutes following administration.

52. A formulation for in situ release in an animal as claimed in claim 51 in which the progesterone and the cyclodextrin formulation on delivery effects a progesterone blood serum level directed to controlling oestrus in cattle of 2-8 ng/ml within 100 minutes following administration and wherein continuous progesterone delivery is required to achieve and maintain said progesterone blood serum level for the duration of dosing (until cessation of administration of the progesterone formulation) for 8 or 10 days in cattle, depending on the delivery regime being implemented.

53. A formulation for in situ release in an animal as claimed in claim 52 in which the progesterone blood serum level is targeted to effect a rapid return to basal progesterone serum levels within 18-24 hours of cessation of administration of the progesterone formulation.

54. A formulation for in situ release in an animal as claimed in claim 52 in which the progesterone blood serum level is targeted to be within the range from 0-2 ng/ml and preferably less than 1 ng/ml within 6 hours of cessation of administration of the progesterone formulation and where there is no endogenous progesterone.

55. A formulation for in situ release in an animal as claimed in claim 52 in which the progesterone and the cyclodextrin formulation on delivery effects a progesterone blood serum level directed to controlling oestrus in cattle of 2-8 ng/ml within 100 minutes following administration to effect coincidence in the delivery regime with the time of first release of the oestrogen and cyclodextrin formulation for in situ release in an animal, the formulation including at least one active component as herein defined for affecting a biological function associated with reproductive processes, in combination with at least one facilitating transfer agent as herein defined, and optionally one or more excipients; said facilitating transfer agent including a cyclodextrin, including a suitable cyclodextrin derivative displaying the preferred properties, a solvent, which includes as an active component an oestrogen or derivative thereof, and a cyclodextrin, including a suitable cyclodextrin derivative displaying the preferred properties, as a facilitating transfer agent, in which the oestrogen or derivative thereof and the cyclodextrin occurs in a ratio of between 1:8 and 1:35 active:facilitating transfer agent, in which the oestrogen and the cyclodextrin formulation on delivery effects a spike in blood serum levels exceeding 130 pg/ml in the time range of 120-180 minutes following administration.

56. A formulation for in situ release in an animal as claimed in claim 28 in which the oestrogen or derivative thereof and the cyclodextrin occurs in a ratio of between 1:8 and 1:35 active:facilitating transfer agent.

57. A formulation for in situ release in an animal as claimed in claim 28 in which the oestrogen or derivative thereof and the cyclodextrin occurs in a ratio of between 1:15 and 1:25 active:facilitating transfer agent.

58. A formulation for in situ release in an animal as claimed in claim 56 in which the oestrogen and the cyclodextrin formulation on delivery effects a spike in blood serum levels exceeding 130 pg/ml in the time range of 120-180 minutes following administration.

59. A formulation for in situ release in an animal as claimed in claim 56 in which the oestrogen in the form of oestradiol 17β when combined with cyclodextrin HPβCD as the facilitating transfer agent is available for use in a delivery regime for synchronising oestrus in an animal at a per unit dose within the range ≧0.5 MG to ≧7 mg of oestradiol.

60. A formulation for in situ release in an animal as claimed in claim 56 in which the oestrogen in the form of oestradiol 17β when combined with cyclodextrin HPβCD as the facilitating transfer agent is available for use in a delivery regime for synchronising oestrus in an animal at a per unit dose of 2 mg oestradiol 17β.

61. A formulation for in situ release in an animal as claimed in claim 20 in which the oestrogen active is oestradiol benzoate.

62. A formulation for in situ release in an animal as claimed in claim 61 in which the oestrogen in the form of oestradiol benzoate when combined with cyclodextrin HPβCD as the facilitating transfer agent is available for use in a delivery regime for synchronising oestrus in an animal at a per unit dose within the range ≧0.9 mg to ≦10 mg of oestradiol benzoate.

63. A formulation for in situ release in an animal as claimed in claim 22 in which an oestrogen active is released more than once.

64. A formulation for in situ release in an animal as claimed in claim 63 in which where the oestrogen active is oestradiol benzoate a first release includes 7 mg of oestradiol per unit dose and a second release includes 2 mg of oestradiol per unit dose when combined with a preferred facilitating transfer agent (such as cyclodextrin HPβCD).

65. A method for affecting a biological function consisting of automated release of active components, the method including an administration regime consisting of at least a first delivery phase for the release of at least a first active component, as well as a second delivery phase for release of at least a second active component, each phase consisting of parameters including one or more of release time, duration, magnitude; the release quantity versus time profiles on said two delivery phases differing, and wherein said first and second active components co-operate to achieved a desired outcome.

66. A method for affecting a biological function as claimed in claim 65 in which the biological function is a reproductive function.

67. A method for affecting a biological function as claimed in claim 66 in which the reproductive function is the synchronisation of oestrus.

68. A method for affecting a biological function consisting of automated release of active components administered in preferred doses via the vaginal route to ensure effective transmucosal absorption of the active required to obtain preferred levels of the active in blood serum for preferred periods.

69. A method for affecting a biological function as claimed in claim 65 in which the first active component is progesterone or derivative thereof.

70. A method for affecting a biological function as claimed in claim 65 in which the second active component is an oestrogen or derivative thereof.

71. A method for affecting a biological function as claimed in claim 65 in which the first delivery phase delivering progesterone or a derivative thereof effects the start of a continuous release profile.

72. A method for affecting a biological function as claimed in claim 65 in which the second delivery phase delivering oestrogen or a derivative thereof effects a release profile of an initial spike followed by one or more spikes after a long interval.

73. A method for affecting a biological function as claimed in claim 72 the second delivery phase delivering oestrogen or a derivative thereof to effect a release profile of an initial spike is administered approximately 2 hours following t=0 on the time-line, whilst to effect a release profile of one or more spikes after a long interval a second spike is administered on or about day nine (9) on the time line.

74. A method as claimed in claim 71 and 72 in which either or both the active progesterone component and the active oestrogen component is delivered in the form of a formulation for in situ release in an animal including at least one active component as herein defined for affecting a biological function associated with reproductive processes, in combination with at least one facilitating transfer agent as herein defined, and optionally one or more excipients; said facilitating transfer agent including a cyclodextrin, including a suitable cyclodextrin derivative displaying the preferred properties, a solvent.

75. A method as claimed in claim 72 in which the active oestrogen component is delivered in a substantially solid form.

76. A method as claimed in claim 74 in which the active oestrogen component is oestradiol 17β or oestradiol benzoate.

77. A method as claimed in either claim 69 wherein to effect transmucosal absorption of the active required to obtain preferred levels of the active in blood serum for preferred periods the active is used in conjunction with at least one facilitating transfer agent.

78. A method as claimed in claim 77 wherein the at least one facilitating transfer agent is available for use in any of the following forms: a powder, a tablet or capsule, a gel, a liquid, a paste, suspensions of varying viscosities, a gas, when said formulation is administered according to the method of claim 65.

79. A method as claimed in claim 78 wherein the at least one facilitating transfer agent is a solvent including at least one of water, an alcohol, a glycol, an organic chemical.

80. A method as claimed in claim 69 wherein when the facilitating transfer agent is an alcohol and the active is a progesterone said alcohol includes at least one of benzyl alcohol, marlophen NP3, propylene glycol P1000, Ethanol and 2-phenylethanol.

81. A method as claimed in claim 78 wherein the at least one facilitating transfer agent is a cyclodextrin, a suitable cyclodextrin derivative displaying the preferred properties, or a substitute compound displaying the preferred properties.

82. A method as claimed in claim 81 wherein when the at least one facilitating transfer agent is a cyclodextrin said cyclodextrin and/or a derivative thereof includes gamma cyclodextrin, beta cyclodextrin, hydroxypropyl β cyclodextrin (HPβCD).

83. A method as claimed in claim 76 wherein when using oestradiol 17β (with a cyclodextrin carrier) for synchronising oestrus in cows, the preferred cyclodextrin facilitating transfer agent is hydroxpropyl 17β-cyclodextrin.

84. A method as claimed in claim 83 wherein the cyclodextrin factitating transfer agent encapsulates/complexes the oestradiol 17 pactive to improve its transferability across membranes.

85. A method as claimed in claim 84 in which the oestradiol 17-β active to cyclodextrin facilitating transfer agent is within the inclusive range of 1:8 to 1:35 (active:facilitating transfer agent) weight for weight.

86. A method as claimed in claim 83 wherein for synchronising oestrus in cows the oestradiol 17β component is within the range ≧0.5 mg to ≧7 mg of oestradiol encased in cyclodextrin.

87. A method as claimed in claim 84 wherein when the cyclodextrin factitating transfer agent is hydroxpropyl 17β-cyclodextrin, efficacious results have been obtained when the oestradiol 17-β formulation is in solid (a tablet) or fluid form.

88. A method as claimed in claim 72 wherein each spike reflects the period the administered formulation used to achieve the desired outcome requires blood serum levels of the active oestrogen component is to be maintained for.

89. A method as claimed in claim 88 wherein the administered formulation used to achieve the desired outcome requires blood serum levels of the oestrogenic active to be maintained at a peak lasting approximately one hour as opposed to maintaining blood serum levels for at least 24 hours.

90. A method as claimed in claim 89 wherein the administered formulation used creates peak plasma concentrations exceeding 130 pg/ml in the time range of 120-180 minutes following administration.

91. A method as claimed in claim 89 where a peak plasma concentration of active falls in the inclusive range 130-180 pg/ml at 100-130 minutes after administration with a 1 mg dose, in the inclusive range 180 to >250 pg at 120-150 minutes following administration with a 2 mg dose, or values extrapolable therefrom for doses of substantially the 1-2 mg range.

92. A method as claimed in claim 89 wherein alteration of the time interval to peak plasma concentrations of active and observed plasma concentration is attained by increasing either or both the amount of the oestrogenic active, and increasing the amount of cyclodextrin.

93. A method for affecting a biological function as claimed in claim 71 in which the first active component is released to maintain substantially a plateau of plasma serum concentration of said first active component throughout the administered delivery.

94. A method for affecting a biological function as claimed in claim 93 in which the active progesterone component is released at substantially regular intervals, the frequency being not substantially longer than the estimated half-life of the in plasma serum.

95. A method for affecting a biological function as claimed in claim 93 in which the active progesterone component is released at substantially regular intervals, the frequency being on average substantially 30-35 minutes apart.

96. A method for affecting a biological function as claimed in claim 95 in which the active progesterone component is released for at least 10 days for a method having a 12 day administered delivery.

97. A method for affecting a biological function as claimed in claim 95 in which the active progesterone component is released for 7-8 days of a ten-day administered delivery.

98. A method for affecting a biological function as claimed in claim 96 wherein the volume of progesterone available for release into the animal over a 10 or 12 day administered delivery ranges between 10 mls to 40 mls of solution.

99. A method for affecting a biological function as claimed in claim 65 in which the administration regime consists of a third delivery phase for the release of a third active component, where the first active component is an oestrogen or derivative thereof, the second active component is a progesterone or derivative thereof and the third active component is a prostaglandin or derivative thereof.

100. A method for affecting a biological function as claimed in claim 99 in which the third delivery phase delivering a prostaglandin or a derivative thereof effects a release profile of a single spike occurring after a first spike of the second active component, but before the second spike of the second active component.

101. A method for affecting a biological function as claimed in claim 99 in which the administration regime is such that the oestrogen active is released according to the release profile in which the second delivery phase delivering oestrogen or a derivative thereof effects a release profile of an initial spike followed by one or more spikes after a long interval, the progesterone active is released according to the release profile in which the first delivery phase delivering progesterone or a derivative thereof effects the start of a continuous release profile, and the prostaglandic active is released according to the release profile in which the third delivery phase delivering a prostaglandin or a derivative thereof effects a release profile of a single spike occurring after a first spike of the second active component, but before the second spike of the second active component.

102. A method as claimed in claim 23 in which the active components are released intravaginally.

103. A method for affecting a biological function as claimed in claim 22 in which the biological function includes any one of affecting digestion, affecting the control of parasites, affecting growth, altering nutritional status, or response to a medicine.

104. A method as claimed in claim 103 in which the active components are released within the digestive tract.

105. A method for controlling a biological function by the concurrent operation of multiple delivery phases each directed to the release of a formulation including at least one active, the delivery being in situ and at substantially the same site, and delivered autonomously from a single arrangement in which one or more of the following parameters of release time, duration, magnitude is controlled in said regime.

106. A method for controlling a biological function as claimed in claim 105 in which the biological function is reproductive.

107. A method for controlling a biological function as claimed in claim 105 in which said formulation for in situ release in an animal includes at least one active component as herein defined for affecting a biological function associated with reproductive processes, in combination with at least one facilitating transfer agent as herein defined, and optionally one or more excipients; said facilitating transfer agent including a cyclodextrin, including a suitable cyclodextrin derivative displaying the preferred properties, a solvent.

108. A method for controlling a biological function as claimed in claim 105 in which a single arrangement for autonomous delivery is a device of the type including a body, the body capable of housing delivery apparatus capable of actively being controlled to autonomously deliver at least one substance into a cavity, said delivery apparatus including dedicated pressure systems to deliver the formulations from independent reservoirs via associated outlet(s), said formulations ranging in form from substantially fluid to substantially solid, the device also including programmable control means capable of initiating and regulating delivery of the formulations in accordance with a preferred delivery regime, the body further including retention apparatus capable of effecting retention of the device within the cavity.

109. A method for controlling a biological function as claimed in claim 105 in which the biological function includes any one of affecting digestion, affecting the control of parasites, affecting growth, altering nutritional status, medicinal.

110. A method for controlling a biological function as claimed in claim 105 in which said formulation(s) capable of being delivered to the animal in situ includes at least one of a parasiticide or insecticide, a vitamin, mineral, or nutritional supplement, a medicine, a prophylactic agent.

111. Reproductive processes in an animal controlled as claimed in claim 65 using a delivery device of the type including a body, the body capable of housing delivery apparatus capable of actively being controlled to autonomously deliver at least one substance into a cavity, said delivery apparatus including dedicated pressure systems to deliver formulations from independent reservoirs via associated outlet(s), said formulations ranging in form from substantially fluid to substantially solid, the device also including programmable control means capable of initiating and regulating delivery of the formulations in accordance with a preferred delivery regime, the body further including retention apparatus capable of effecting retention of the device within the cavity.

112. A biological function controlled as claimed in claim 105.

113. A delivery device of the type including a body, the body capable of housing delivery apparatus capable of actively being controlled to autonomously deliver at least one formulation into a cavity, said delivery apparatus including dedicated pressure systems to deliver the formulations from independent reservoirs via associated outlet(s), said formulations ranging in form from substantially fluid to substantially solid, the device also including programmable control means capable of initiating and regulating delivery of the formulations in accordance with a preferred delivery regime, the body further including retention apparatus capable of effecting retention of the device within the cavity, said programmable control means programmed to implement a method as claimed in claim 65.

114. A delivery device as claimed in claim 113 wherein the device is adapted to be retained in the animal in the preferred delivery site for at least the duration of the delivery regime, including being externally applied to the animal and being attached to the delivery device located internally of the animal; or being internal structures on or associated with an internally located delivery device; or involve an external delivery device having external retention apparatus, but with delivery conduits inserted into the animal.

115. A delivery device as claimed in claim 114 in which said device is used for effecting control of a biological function or a stage thereof.

116. A delivery device as claimed in claim 115 wherein said device is used for effecting oestrus synchronisation.

117. A delivery device as claimed in claim 116 wherein the device is an intravaginal delivery device, adapted to deliver the required hormones in required doses at required times into the anterior vagina of the animal for which synchronised oestrus is required.

118. A delivery device as claimed in claim 117 in which said device is used for effecting oestrus synchronisation, particularly for: single round synchrony of any one of lactating, non-lactating, cycling, anoestrus, dairy or beef cows and heifers, for cows or heifers intended for breeding, for cows or heifers intended for fixed time planned insemination for cows and heifers intended to be artificially inseminated.

119. A delivery device of the type including a body, the body capable of housing delivery apparatus capable of actively being controlled to autonomously deliver at least one formulation into a cavity, said delivery apparatus including dedicated pressure systems to deliver the formulations from independent reservoirs via associated outlet(s), said formulations ranging in form from substantially fluid to substantially solid, the device also including programmable control means capable of initiating and regulating delivery of the formulations in accordance with a preferred delivery regime, the body further including retention apparatus capable of effecting retention of the device within the cavity, said device containing at least one formulation as claimed in claim 20.

120. A delivery device for delivering preferred formulation(s) as claimed in claim 119 wherein where there are multiple outlets delivering the same active, the concentration of the formulation may vary, additional compounds may be added to the separate formulations to effect delivery of these additives at precise times in combination with the active, or the duration of a particular delivery may be adapted to coincide with a particular stage of the biological function being controlled.

121. A delivery device for delivering preferred formulation(s) as claimed in claim 120 wherein a single formulation may be delivered from separate outlets at separate times, whilst at least one other formulation may be delivered from other outlets at the same time, or at timed intervals before, during or after delivery of the first or additional formulations, or other combinations as simply determined by the formulation(s) used and the biological function being controlled.

122. A delivery device as claimed in claim 119 wherein the said programmable control means is programmed to effect initiation and/or regulation of delivery of said formulations in either or both in sequence and in unison, from the delivery device to effect control of one or more stages of a known biological function to effect a desired physiological response.

123. A delivery device as claimed in claim 122 for delivering preferred formulation(s) wherein use of programmable control means for initiating and regulating delivery of the formulation(s) is electric in operation and includes:
a power source,
a microprocessor able to run software for determining and controlling the delivery of a dose by the substance delivery device according to a predetermined delivery regime,
a printed circuit board including components for effecting operation of either or both of resistors and an electromagnetic coil in response to the software being run by the microprocessor, their operation resulting in autonomous delivery of at least one substance from at least one said reservoir in accordance with the aforesaid predetermined delivery regime, and
a switch to activate the substance delivery device.

124. A delivery device for controlling a biological function as claimed in claim 119 in which said at least one formulation having efficacy in effecting control of at least one stage of a preferred biological function and having improved permeation to effect desired bioavailability of at least one active(s) maintained at a preferred level for a preferred period of time, said formulations adapted to be delivered via said delivery device retained in location at a specific site for at least the delivery period, to achieve the outcome required, and characterised by use of formulations in either or both substantially solid and substantially fluid form delivered autonomously at a single site.

125. A delivery device as claimed in claim 124 in which said at least one formulation(s) is delivered in accordance with said preferred delivery regime in predetermined concentration(s), in predetermined quantity(s), delivered at predetermined time intervals and over predetermined period(s).

126. A delivery device as claimed in claim 125 wherein said at least one formulation delivered from the device is directed specifically to synchronising oestrus.

127. A delivery device as claimed in claim 126 wherein said at least one formulation delivered from the device is directed to synchronising oestrus in A cow.

128. A delivery device as claimed in claim 126 wherein said at least one formulation delivered from the device includes an active from a list including an oestrogen, a progesterone, a prostaglandin, or a derivative or an analogue thereof.

129. A delivery device as claimed in claim 128 wherein said at least one formulation comprises progesterone as an active for use as an ovarian suppressant and includes 2 grams progesterone provided in 7 millilitres of solution, wherein propylene glycol is used as a co-solvent and phenylethanol as a solvent, with β-cyclodextrin as a complexing agent.

130. A delivery device as claimed in claim 128 wherein said at least one formulation comprises progesterone as an active for use as an ovarian suppressant and includes progesterone provided in substantially solid form, with P-cyclodextrin as a complexing agent.

131. A delivery device as claimed in claim 128 wherein said at least one formulation comprises 17 β-oestradiol as an oestrogenic active and includes 7 milligrams of the oestradiol provided in one 100 milligram tablet, with β-cyclodextrin as a complexing agent, cellulose as a binding agent, colloidal silica as a flowing agent, and magnesium stearate as a lubricating agent and optionally a colourant, as a first oestradiol formulation.

132. A delivery device as claimed in claim 128 wherein said at least one formulation comprises 17 β-oestradiol as an oestrogenic active and includes 2 milligrams of the oestradiol provided in one 60 mg, β-cyclodextrin as a complexing agent, cellulose as a binding agent, colloidal silica as a flowing agent, and magnesium stearate as a lubricating agent and optionally a colourant, as a second oestradiol formulation.

133. A delivery device as claimed in claim 128 wherein said at least one formulation comprises sodium cloprostenol sodium as a prostaglandin active for use as for use as a luteolytic agent and includes 240 micrograms of the sodium cloprostenol provided in one 60 mg tablet, cellulose as a binding agent, colloidal silica as a flowing agent, and magnesium stearate as a lubricating agent.

134. A delivery device as claimed in claim 128 wherein a single delivery device is adapted to deliver all three the actives such that a total of 1.10 g progesterone is delivered as a series of pulsatile doses, oestradiol is delivered as a two single doses of 2.00 mg each and 1.00 mg of sodium cloprostenol is delivered as a single dose.

135. A delivery device as claimed in claim 124 wherein the single site the delivery device is located is the anterior vagina of the cow and delivers the active in said at least one formulation to the vaginal mucosa via a pressure/pumping delivery system.

136. A delivery device as claimed in claim 135 wherein the oestradiol and prostaglandin formulations are delivered via a pressure/pumping delivery system are delivered as single doses through a pot release and/or an automated syringe mechanism.

137. A delivery device as claimed in claim 131 wherein single doses of both the first and second oestradiol formulations and the prostaglandin formulation delivered via the pot releases, are required to effect the desired synchrony of oestrus where the target animal is a cow.

138. A delivery device as claimed in claim 129 wherein the progesterone formulation is delivered from either or both a collapsible bellows reservoir and a conduit.

139. A delivery device as claimed in claim 130 wherein the progesterone formulation is delivered.from a conduit where said progesterone is delivered in substantially solid form relying on passive delivery through process of dissolution in fluids of the animal's body cavity.

140. A delivery device as claimed in claim 138 wherein the progesterone formulation is delivered from a conduit where said progesterone is delivered in substantially fluid form relying on controlled active delivery from the delivery device.

141. A method of determining a delivery regime for implementation in effecting control of a biological function, or one or more stages thereof, using formulations or a series of formulations as claimed in claim 1, said method including the steps of: determining the preferred formulations instrumental in effecting control of the biological function or stages thereof; and determining delivery phases required to effect release of one or more of the preferred formulation(s) of predetermined concentration(s), in predetermined quantity(s), at predetermined time(s) and over predetermined period(s) for a delivery period; and effecting delivery of the formulations in accordance with the delivery phases from a substance delivery device, said delivery device being adapted to be retained in location in an animal for at least the delivery period, being adapted to house the formulations and including control and delivery apparatus to effect controlled release of the formulations in accordance with the delivery regime, the method characterised by the delivery regime effecting control of the biological function through the autonomous delivery of the formulations, from the delivery device located in situ, at a single site in the animal's body to effect a desired physiological response in an animal for which it is intended to be used.

142. A method of determining a delivery regime for implementation in effecting control of a biological function or one or more stages thereof as claimed in claim 123 wherein said delivery regime is effected via pre-programming and control via programmable electronic control means included in the delivery device from which the formulations are released in situ.

143. A method of determining a delivery regime for implementation in effecting control of a biological function or one or more stages thereof as claimed in claim 142 wherein the programmable control means effects implementation of the delivery regime by effecting one or more of:
activation, initiation and regulation of delivery of the formulation(s) from the preferred delivery device that houses the formulations in situ,
delivery of the preferred actives formulation(s) in sequence and/or in unison
delivery of the preferred actives formulation(s) at preferred times,
delivery of the preferred actives formulation(s) from either or both specific reservoirs and specific outlets of the delivery device,
delivery of the preferred actives formulation(s) for varying lengths of time,
regulation within the sequence of individual aspects of the formulation(s) delivery including the duration and/or outlet opening and hence quantity of formulations delivered,
signalling of the endpoint of one delivery and the start of another,
simultaneous delivery of one or more specific formulations as and when required
delivery of the preferred actives formulation(s) from one or more outlets of the delivery device, at the same time.

144. A method of determining a delivery regime for implementation in effecting control of a biological function or one or more stages thereof as claimed in claim 143 wherein the delivery regime is predetermined and the delivery system pre-programmed and pre-calibrated to deliver the required formulations according to a required delivery phase within the overall delivery regime.

145. A method of determining a delivery regime for implementation in effecting control of a biological function or one or more stages thereof as claimed in claim 143 wherein the delivery regime provides for one or more modes of delivery including a single unit delivery, continuous delivery, continuous pulsatile delivery, intermittent pulsatile delivery, passive delivery of the formulations.

146. A method of determining a delivery regime for implementation in effecting control of a biological function or one or more stages thereof as claimed in claim 145 wherein where the delivery regime provides for one or more modes of delivery the formulations are delivered in solid (tablet or capsule), liquid (including gels, solutions, sprays), suspension (pastes or forms having various viscosities) or gaseous form, determined by the permeability, desired speed of transfer across membranes and required bioavailability.

147. A method of determining a delivery regime for implementation in effecting control of a biological function or one or more stages thereof as claimed in claim 141 wherein where the delivery regime provides for delivery of formulations in which the facilitating transfer agent and the active are: complexed to form a specific premixed formulation; present within the same (solid) formulation but not complexed; released separately at or about the same time and mixed to effect the formulation during the release process; released separately but released in the same target location at or about the same time so that mixing is enabled in the vicinity of the release zone to effect the formulation, and wherein either or both the active and facilitating transfer agent are in substantially dry form and substantially fluid form when mixed to effect the formulation released in situ into the animal.

148. A method of determining a delivery regime for implementation in effecting control of a biological function or one or more stages thereof as claimed in claim 143 wherein the programmable control means is required to effect implementation of the delivery regime by via use of a microprocessor capable of running dose control software.

149. A method of determining a delivery regime for implementation in effecting control of a biological function or one or more stages thereof as claimed in claim 148 wherein the implementation of the delivery regime via use of a microprocessor capable of running dose control software for synchronizing oestrus in cattle, requires dose control to be exerted over the time at which single unit doses are delivered for the oestrogen and prostaglandin formulations and over the duration and the dose volume of a continuous series of pulsatile doses for delivery of the progesterone formulation.

150. A method of determining a delivery regime for implementation in effecting control of a biological function or one or more stages thereof as claimed in claim 149 wherein delivery of the oestradiol and prostaglandin formulations as single doses is effected through a pot release and/or an automated plunger/syringe mechanism, there being two such pot releases of the oestradiol and one of prostaglandin, as required to effect the desired hormone regime to effect synchrony of oestrus in the target animal.

151. A method of determining a delivery regime for implementation in effecting control of a biological function or one or more stages thereof as claimed in claim 149 wherein delivery of progesterone in solution as a continuous programmed series of pulsatile doses is effected by the solution containing progesterone being retained within a collapsible reservoir with the solution presented to the inlet of a pump to which the reservoir is attached, said delivery from the said reservoir being effected by electronic control means including a microprocessor capable of running dose control software.

152. A method of determining a delivery regime for implementation in effecting control of a biological function or one or more stages thereof as claimed in claim 151 wherein the dose control software is effects delivery as a continuous series of pulsatile doses with interdose intervals of less than the metabolic rate of the active to effect a high probability of elevating and maintaining p4 blood serum values (levels in serum 4 days after administration) of in situ progesterone above a required minimum threshold of 2 ng/ml.

153. A method of determining a delivery regime for implementation in effecting control of a biological function or one or more stages thereof as claimed in claim 152 wherein to maintain blood serum levels of in situ progesterone above a required minimum threshold of 2 ng/ml, the dose control software is programmed to compensate for the decreasing volume of solution delivered per dose over time, by increasing the number of times the micropump operates during a given period.

154. An animal whose biological function is being controlled according to a method as claimed in claim 65.

155. An animal whose biological function or a stage thereof is being controlled through use of delivery device means.

156. An animal to whom formulations of claim 1 are delivered.

157. A method of producing an animal in a state of ovulation using formulations as claimed in claim 1.

158. A method of producing an animal in a state of ovulation using formulations as claimed in claim 1 wherein the animal is a cow.

159. A method of producing an animal in a state of ovulation as claimed in claim 158 to stimulate and synchronise oestrus in cycling or non-cycling cows or heifers intended for breeding, for fixed time planned insemination and for cows and heifers that are to be artificially inseminated.

160. A method of producing an animal in a state of ovulation as claimed in claim 159 wherein administration of formulations occurs over a 12 day delivery regime for cycling or anoestrus, lactating or non lactating dairy or beef cows and heifers, said method including the steps of: insertion of a preferred delivery device into the anterior vagina of the animal on day one followed by administration of approximately 42 mg 5% progesterone solution dosed 2 hourly to initially elevate the levels of progesterone to effect control on the current fertility status of all animals being treated and this is the first step in resetting the follicular waves, and followed by a spike release of 6.8 mg Oestradiol Benzoate, such that these treatments in synergy have the objective of suppressing follicular waves; and on days 2 to 10 administration of 42 mg 5% progesterone solution dosed 2 hourly, followed by a spike release of 240 mcg prostaglandin (Cloprostenol Sodium) on day 10, which is luteolytic and prevents the animal from producing any endogenous progesterone and effects regression of a corpus luteum if present, and ceasing progesterone delivery, and delivering a spike release of 0.9 mg Oestradiol Benzoate on day 11, such that the abrupt cessation of progesterone release, as well as an oestradiol pulse, are intended to initiate FSH/LH surges leading to follicle maturation and ovulation, and removal of the intravaginal delivery device and insemination of all cows in the treatment group.

161. A method of producing an animal in a state of ovulation as claimed in claim 159 wherein administration of formulations occurs over a 10 day delivery regime for cycling or anoestrus, lactating or non lactating dairy or beef cows and heifers, said method including the steps of: insertion of a preferred delivery device in to the anterior vagina of the animal on day one followed by administration of progesterone release 20 minutes after device activation to initially elevate the levels of progesterone to effect control on the current fertility status of all animals being treated being the first step in resetting the follicular waves, and continuing with pulses at a frequency to effect maintenance of blood progesterone >2 ng/mL for 8 days; and release of oestradiol within 120 minutes of device activation to produce a spike of >25 pg/mL blood oestradiol, such that these treatments in synergy have the objective of suppressing follicular waves; and on day 7 release of a single pulse of 1.00 mg prostaglandin cloprostenol sodium which is luteolytic and prevents the animal from producing any endogenous progesterone and effects regression of a corpus luteum if present; and ceasing progesterone delivery at the end of day 8; and on day 9 release of a second single spike release of oestradiol (2.00 mg), such that the abrupt cessation of progesterone release, as well as an oestradiol pulse, are intended to initiate FSH/LH surges leading to follicle maturation and ovulation, and removal of the intravaginal delivery device and insemination of all cows in the treatment group.

162. A method of producing an animal in a state of ovulation as claimed in either or both claim 160 and claim 161 wherein a pronounced oestradiol spike (for short duration), total bioavailability of the oestradiol, or the period above a critical value, is positively correlated with clinical efficacy for either follicular atresia or stimulation of oestrus.

163. A method of producing an animal in a state of ovulation as claimed in either or both claim 160 and claim 161 claim wherein the oestrogen active (oestradiol) is used to ensure the ovulatory follicle after ten, or eight days respectively (depending on the programme), of progesterone therapy is an actively growing healthy follicle producing an ovum consistently capable of being fertilised and initiating pregnancy.

164. A method of producing an animal in a state of ovulation as claimed in either or both claim 160 and claim 161 wherein the progesterone formulation used ensures follicular waves are initiated in anoestrus cows that do not display these and resets the follicular waves in cycling cows.

165. An animal prepared in a state of ovulation resulting from a method as claimed in claim 65.

166. A delivery device of the type including a body, the body capable of housing delivery apparatus capable of actively being controlled to autonomously deliver at least one formulation into a cavity, said delivery apparatus including dedicated pressure systems to deliver the formulations from independent reservoirs via associated outlet(s), said formulations ranging in form from substantially fluid to substantially solid, the device also including programmable control means capable of initiating and regulating delivery of the formulations in accordance with a preferred delivery regime, the body further including retention apparatus capable of effecting retention of the device within the cavity, said programmable control means programmed to release a series of formulations as claimed in claim 1, according to predetermined parameters including one or more of: delay to release, frequency of release, release duration, and period over which release functions occur.

167. A pre-fertilised viable egg of an animal resulting from the synchronisation of oestrus from the controlled delivery in situ of a series of formulations as claimed in claim 1.

168. A pre-fertilised viable egg of an animal resulting from the synchronisation of oestrus according to the method of claim 67.

169. A pre-fertilised viable egg of an animal resulting from a method as claimed in claim 157.

170. Formulations containing one or more of the group containing: progesterone and derivatives, oestrogen and derivatives, and prostaglandin and derivatives; in combination with at least one cyclodextrin and/or derivatives, adopted for use in the method of claim 68.

171. Formulations containing one or more of the group containing: progesterone and derivatives, oestrogen and derivatives, and prostaglandin and derivatives; in combination with at least one cyclodextrin and/or derivatives, when used according to the method of claim 68.
